(19) United States
(12) Patent Application Publication
(10) Pub. No.: US 2006/0093597 A1
(43) Pub. Date: May 4, 2006

(54) Methods of treating involuntary facial spasms and facial wrinkles

(76) Inventor: Alex Zhu, New York, NY (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/525,630

(22) PCT Filed: Aug. 25, 2003
(86) PCT No.: PCT/US03/26676

§ 371 Date: Oct. 24, 2005

Related U.S. Application Data
(63) Continuation-in-part of application No. 10/365,108, filed on Feb. 12, 2003.
(60) Provisional application No. 60/405,779, filed on Aug. 23, 2002.

(51) Int. Cl. A61K 38/54; A61K 38/14; A61K 31/704; A61K 36/28; A61K 33/06; A61K 31/65

(52) U.S. Cl. 424/94.2; 424/682; 424/764; 424/779; 514/28; 514/37; 514/39; 514/38; 514/154; 514/8

(57) ABSTRACT

The invention describes antibiotics, muscle relaxants and plant extracts that have neuromuscular blockade effects as well as methods of use thereof. These compounds can be used in the same clinical settings as botulinum toxin and may be used topically, thereby providing an advantage over botulinum toxin in terms of application and ease of use. The compounds can be used in pharmaceutical compositions for the treatment of involuntary muscle spasms and neuropathic pain and in cosmetic compositions for the treatment of facial wrinkles. Also provided are kits useful for therapeutic and/or cosmetic applications.

### BACKGROUND OF THE INVENTION

Several compounds are used to modulate the activity at neuromuscular junctions and display neuromuscular blockade effects. One such compound is botulinum toxin, which blocks the release of acetylcholine from the neuromuscular junction, and has been applied to a variety of therapeutic and cosmetic conditions. These applications include, but are not limited to, ocular disorders; dystonia, bleopharospasm, hemifacial spasm, synkinesis, and the involuntary facial muscle spasms caused by these disorders; gastrointestinal disorders; management of pain; neuropathic pain and treatment of facial wrinkles. In all these cases, the toxin needs to be injected in the area where symptoms occur in order to exert a therapeutic effect.

The present invention can be utilized in a variety of therapeutic and cosmetic conditions where botulinum toxin is currently used (Binder et al. Dis Mon 48: 323-335, 2002; Epperson, Ala Med 65: 49-50, 1995; Khawaja et al. Int J Dermatol 40: 311-317, 2001; Mendez-Eastman, Plast Surg Nurs 20: 60-65, 2000; Verheyden and Blitzer, Dis Mon 48: 357-366, 2002). Additionally, synkinesis, a common phenomenon associated with facial paralysis, and facial wrinkles, is utilized as an example of a botulinum toxin-treatable condition to demonstrate novelty and utility of the invention.

Synkinesis

Approximately 7,000 neuron cell bodies make up the facial nerve, each of which innervates approximately 25 muscle fibers. The axons are surrounded by myelin, produced by the Schwann cells surrounding the axons. Three membranes comprise the nerve sheath. The epineurium is the outer covering, composed of loose areolar tissue, which separates the fascicles and holds them together. The perineurium is the next more inner layer. This is a dense layer of cells that are metabolically active and function as a diffusion barrier. The perineurium provides considerable strength to the nerve sheath. The individual nerve fibers are then each surrounded by the endoneurium.

Involuntary synkinesis between the orbicularis oculi (eye) and orbicularis oris (mouth) muscle is a very common side effect in patients recovering from facial nerve paralysis. It results from an aberrant connection of the motor fibers originally innervating orbicularis oris to the orbicularis oculi, causing lid closure whenever patients smile, talk or eat. If severe, this can lead to social embarrassment and functional visual loss.

When the facial nerve is damaged by surgery or viral infection, each of the nerve fibers needs to regrow. However, the appropriate nerve fiber ends do not always grow and connect correctly, resulting in a “crossed wires” phenomenon. This results in the misdirection of nerve impulses at the site of the nerve injury. Synkinesis varies in severity from mild to severe. In its worst form, it can result in uncontrollable movement of the facial muscles on the affected side during any attempted expression. The affected side of the face may feel tight as the result of the uncontrolled muscle contractions (spasms).

Current Medical Treatment

Medical Therapy:

Currently, the only medical treatment for synkinesis is the injection of botulinum toxin, currently manufactured and marketed by Allergan, Inc. under the brand name Botox (Armstrong et al. Clin Otolarylngol 21: 15-20,1 1996; Laskawi et al. Eur Arch Otorhinolaryngol S195-S199, 1994; Mountain et al. Clin Otolaryngol 17: 223-224, 1992; Roggenkamper et al. Doc Ophthalmol 86: 395-402, 1994). The use of botulinum toxin as chemical neurectomy offers an effective though not a curative treatment. The toxin is injected subcutaneously in the pretarsal areas in both the upper and lower lids as in blepharospasm. This has the effect of paralyzing the orbicularis and therefore reduces the synkinesis. The treatment takes a few days to work and usually lasts between two to three months. Repeated injection is required when the effect wanes.

Facial muscles have a tendency to become hypertonic (overactive) after paralysis. Weakening or re-paralyzing the muscles with Botox injection may temporarily ease the effects of some synkinesis and hypertonic muscles. Spasms can be reduced along with the pain and discomfort associated with them.

Some patients may benefit from a special form of physical therapy called facial retraining (Diels and Combs, Otolaryngol Clin North Am 30: 727-743, 1997).

Electrical stimulation has been discouraged by most doctors based on mounting evidence that it may be harmful to the nerve's ability to regenerate. Electrical stimulation may result in a mass contraction of the facial muscles thereby producing an undesirable, uncoordinated muscle response.

Surgical Therapy:

Surgical repair of the facial nerve is generally performed in cases of complete paralysis. Presently, three options exist for repair of the facial nerve. Options include direct repair, cable nerve grafting, and nerve substitution techniques (Angeli and Chiossone, Otolaryngol Clin North Am 30: 683-700, 1997). Synkinesis is expected in all cases, regardless of the mode of repair chosen. The best result one can hope for is a House-Brackman grade III. The House-Brackmann grading scale (6 levels) for facial paralysis is used to objectively describe the degree of facial paralysis (House and Brackman, Otolaryngol Head Neck Surg. 93,146-147, 1985).

| Grade | Characteristics |
|---|---|
| I. Normal | Normal facial function in all areas |
| II. Mild dysfunction | Gross |

Slight weakness noticeable on close
inspection
May have slight synkinesis
At rest, normal symmetry and tone

Motion

Forehead - Moderate-to-good function
Eye - Complete closure with minimal
effort
Mouth - Slight asymmetry

| | |
|---|---|
| III. Moderate | Gross |

| dysfunction | Obvious but not disfiguring difference |
|---|---|
| | between the two sides |
| | Noticeable but not severe synkinesis, |
| | contracture, or hemifacial spasm |
| | At rest, normal symmetry and tone |

Motion

| | Forehead - Slight-to-moderate |
|---|---|
| | movement |
| | Eye - Complete closure with effort |
| | Mouth - Slightly weak with maximum |
| | effort |
| IV. Moderately severe | Gross, Obvious weakness and/or |
| dysfunction | disfiguring asymmetry |
| | At rest, normal symmetry and tone |
| | Motion |
| | Forehead - None |
| | Eye - Incomplete closure |
| | Mouth - Asymmetric with maximum |
| | effort |

| | |
|---|---|
| V. Severe dysfunction | Gross |

Only barely perceptible motion
At rest, asymmetry

Motion

Forehead - None
Eye - Incomplete closure
Mouth - Slight movement

| | |
|---|---|
| VI. Total paralysis | No movement |

Donor site morbidity is also expected for the hypoglossal crossover technique (tongue weakness), great auricular harvest (ear numbness), and sural nerve harvest (lateral leg numbness). Complications for each of the procedures can include hematoma and infection.

Neuropathic Pain

Neuropathic pain is associated with injury to the peripheral and/or central nervous system. Several conditions are examples of neuropathic pain including, but not limited to: postherpetic neuralgia, diabetic neuropathy, complex regional pain syndrome, or spinal cord injury. In addition, other pain syndromes including, but not limited to: radiculopathy, migraine headache, and myofascial pain, exhibit clinical and pathophysiologic features consistent with neuropathic pain.

The most common peripheral compressive neuropathy is carpal tunnel syndrome, which causes sensory and motor disturbances in the hand. It occurs when the main nerve to the hand becomes squeezed or pinched at the wrist. Pain, numbness, and weakness of the hand are classic signs of carpel tunnel syndrome. It was estimated that over 10 million Americans are affected by the syndrome.

Current Medical Treatment

No standardized, consistently effective, and conservative treatments are currently available for carpal tunnel syndrome. Treatment approaches commonly used include hand splinting, vitamin B6, steroid injections and oral anti-inflammatory medications. Due to frequent recurrences and advances in severity, many patients may ultimately have to undergo surgical decompression and neurolysis.

Botulinum toxin has been shown to alleviate pains resulted from various neuropathic conditions including carpal tunnel syndrome, although the mechanism for pain relief is not completely understood.

Facial Wrinkles

There are two different types of wrinkles, fine wrinkles and dynamic wrinkles. Traditionally, fine wrinkles are believed to result from aging, sun exposure, and reduction in collagen and elastic fibers of the skin. Deep wrinkles are associated with the build-up of the musculature below the skin surface. When people with healthy skin are making facial expressions, they show dynamic wrinkles around the facial expression muscles. When those muscles are at rest, skin resumes its most smoothness due to elasticity. It is believed, however, that muscles at resting stage are in a slight contractile posture due to base-level/spontaneous quantal release of acetylcholine. Healthy skin looks smooth because elasticity overcomes this mild contractile muscle posture to hide obvious wrinkles. Facial wrinkles are generated presumably by the breakdown of skin support of collagen and elastin fiber due to aging and sun exposure, diminished function of sweat glands and fat pad atrophy beneath the dermis. In addition, years of repeating the facial muscle contraction can thicken the muscle layer and thus deepen the wrinkles.

Current Facial Wrinkle Treatments

Many approaches are taken to reduce the appearance of facial wrinkles based on the understanding of the molecular basis of wrinkle formation. Such treatments include cosmetic products, drug therapy and surgical procedures. For example, many cosmetic products contain alpha hydroxy acids, which may stimulate collagen synthesis. Another common treatment utilizes retinol, a derivative of vitamin A, (or its stronger, prescribed version Retin-A and Renova) which helps collagen production. Antioxidants such as vitamin C and E and coenzyme Q-10 are believed to counteract free radicals, which damage cells and cause aging and have been used in treatments of wrinkles. Recently, the FDA approved cosmetic use of Botox (an extremely diluted form of botulinum toxin) to treat glabellar frown lines.

Botulinum Toxin Therapy

Botulinum toxin blocks the release of acetylcholine from the neuromuscular junction, and has been applied to a variety of cosmetic conditions (Binder et al. Dis Mon 48: 323-335, 2002; Epperson, Ala Med 65: 49-50, 1995; Khawaja et al. Int J Dermatol 40: 311-317, 2001; Mendez-Eastman, Plast Surg Nurs 20: 60-65, 2000; Verheyden and Blitzer, Dis Mon 48: 357-366, 2002). These applications include treatment of facial wrinkles. In such cases, the toxin needs to be injected in the area where symptoms occur in order to exert an effect.

Cosmetic use of botulinum toxin in minimizing facial wrinkles underscores the theory that contractile muscle fiber, which is under the direct control of the neural motor influx, is likely to play an important role in the formation of wrinkles, particularly glabellar frown lines. Locally blocking the motor contraction by botulinum toxin not only minimizes wrinkles but also exerts a “smoothing” effect on the skin's microrelief.

Disadvantages of Botulinum Toxin Therapy

Botulinum toxin, produced by Clostridium botulinuin, consists of a 100-kD heavy chain linked to a 50-kD light chain by a disulfide bond (Singh, Nat Struct Biol 7: 617-619, 2000). The toxin blocks the release of acetylcholine from cholinergic nerve endings by a three-step mechanism. First, the toxin binds to presynaptic terminals. Second, the toxin is internalized by receptor-mediated endocytosis and released into the cytosol. Finally, the light chain enzymatically blocks exocytosis (release) of acetylcholine at the neuromuscular junction.

Complications associated with the botulinum toxin therapy may partially result from the apparent diffusion of the toxin from the injected muscle(s) to adjacent muscles (Khawaja et al. Int J Dermatol 40: 311-317, 2001). When such diffusion occurs, the resulting muscle paralysis can cause double vision or ptosis so severe that sight is obstructed by the drooping eyelid.

It was initially believed, based on the experience with food-borne botulism, that individuals exposed to botulinum toxin did not produce antibodies against the toxin, due to extremely low dosage of the toxin therapeutically used. However, a different picture has emerged from the toxin therapy. It has been observed that some patients, who initially benefited from the toxin, later became insensitive (refractory, resistant) to its use. This insensitivity has been attributed to the development, upon repeated injections with the toxin, of antibodies against the toxin. Jankovic and Schwartz found neutralizing antibodies against the toxin in 5 (37.5%) sera from 14 patients characterized as “non-responders” to botulinum toxin therapy, whereas no antibodies were found in 32 patients characterized as “responders” to the toxin (P<0.0001) (Jankovic and Schwartz, Arch Neurol 48: 1253-1256, 1991). In a group of 20 patients categorized as “maintained response” or as “diminished response” who had been treated for several years with botulinum type A toxin, seven (35%) of the patients were found to have toxin-neutralizing antibodies (Hambleton et al. BMJ 304: 959-960, 1992).

Therefore, long-term use of botulinum toxin, even at very low concentration, is likely to induce toxin-neutralizing antibodies, which will decrease the therapeutic effect. For the same reason, people who are vaccinated against botulinum toxin for preventing botulism cannot benefit from the toxin therapy even if such a medical need arises.

Another inherent drawback for botulinum toxin treatment is that the toxin has to be delivered to the symptomatic site by subcutaneous injection. As a protein of 150 kDa, the toxin is far too large a molecule to penetrate the skin to exert its therapeutic effect.

### SUMMARY OF THE INVENTION

In one aspect, the invention disclosed herein includes a method for localized chemodenervation by topically administering a pharmaceutical composition containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier.

In another aspect, the invention includes a method for localized chemodenervation by topically administering a pharmaceutical composition containing a pharmaceutically effective amount of one or more compounds selected from the group consisting of a magnesium salt, an organic magnesium compound or a plant extract, and a pharmaceutically acceptable carrier.

In yet another aspect, the invention includes a method for treating involuntary facial muscle spasms by topically administering a pharmaceutical composition containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier.

In another aspect, the invention includes a method for treating involuntary facial muscle spasms by topically administering a pharmaceutical composition containing a pharmaceutically effective amount of one or more compounds selected from the group consisting of a magnesium salt, an organic magnesium compound, a plant extract or analogs and derivatives thereof, and a pharmaceutically acceptable carrier.

In yet another aspect, the invention includes a method for reducing facial wrinkles by topically administering a pharmaceutical composition containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier.

In another aspect, the invention includes a method for reducing facial wrinkles by topically administering a pharmaceutical composition containing a pharmaceutically effective amount of one or more compounds selected from the group consisting of a magnesium salt, an organic magnesium compound, a plant extract or analogs and derivatives thereof, and a pharmaceutically acceptable carrier.

In yet another aspect, the invention includes a method for treating or reducing neuropathic pain by topically administering a pharmaceutical composition containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier.

In another aspect, the invention includes a method for treating or reducing neuropathic pain by topically administering a pharmaceutical composition containing a pharmaceutically effective amount of one or more compounds selected from the group consisting of a magnesium salt, an organic magnesium compound, a plant extract or analogs and derivatives thereof, and a pharmaceutically acceptable carrier.

In yet another aspect, the invention includes a pharmaceutical composition for topical administration for localized chemodenervation containing pharmaceutically effective amount of an antibiotic, aminoglycoside antibiotic, polymyxin, tetracycline, lincosamide, muscle relaxant, plant extract or analogs and derivatives thereof in a pharmaceutically acceptable medium compatible with human skin. In a preferred aspect, the pharmaceutical composition further contains a magnesium salt or organic magnesium compound.

In yet another aspect, the invention includes a pharmaceutical composition for topical administration for treating involuntary facial muscle spasms containing effective amount of an antibiotic, aminoglycoside antibiotic, polymyxin, tetracycline, lincosamide, muscle relaxant, plant extract or analogs and derivatives thereof in a medium compatible with human skin. In a preferred aspect, the pharmaceutical composition further contains a magnesium salt or organic magnesium compound.

In yet another aspect, the invention includes a pharmaceutical composition for topical administration for reducing facial wrinkles containing effective amount of an antibiotic, aminoglycoside antibiotic, polymyxin, tetracycline, lincosamide, muscle relaxant, plant extract or analogs and derivatives thereof in a medium compatible with human skin. In a preferred aspect, the pharmaceutical composition further contains a magnesium salt or organic magnesium compound.

In yet another aspect, the invention includes a pharmaceutical composition for topical administration for treating or reducing neuropathic pain containing effective amount of an antibiotic, aminoglycoside antibiotic, polymyxin, tetracycline, lincosamide, muscle relaxant, plant extract or analogs and derivatives thereof in a medium compatible with human skin. In a preferred aspect, the pharmaceutical composition further contains a magnesium salt or organic magnesium compound.

In a further aspect, the invention includes a kit containing a pharmaceutical composition for localized chemodenervation containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier, and instructions for topical administration of the composition.

In yet another aspect, the invention includes a kit containing a pharmaceutical composition for treating involuntary facial muscle spasms containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier, and instructions for topical administration of the composition.

In yet another aspect, the invention includes a kit containing a pharmaceutical composition for reducing facial wrinkles containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier, and instructions for topical administration of the composition.

In yet another aspect, the invention includes a kit containing a pharmaceutical composition for treating or reducing neuropathic pain containing a pharmaceutically effective amount of an antibiotic, aminoglycoside antibiotic, polymyxin, tetracycline, lincosamide, muscle relaxant, plant extract or analogs and derivatives thereof and a pharmaceutically acceptable carrier, and instructions for topical administration of the composition.

The aminoglycoside antibiotic can be amikacin, gentamicin, kanamycin, neomycin, netilmicin, paromomycin, streptomycin, tobramycin or analogs and derivatives thereof. The amount of neomycin can be between about 2.5 mg/g and about 250 mg/g. The lincosamide can be clindamycin, lincomycin or analogs and derivatives thereof. The muscle relaxant can be a non-depolarizing agent or a depolarizing agent or analogs and derivatives thereof. The non-depolarizing agent can be pancuronium, vecuronium, mivacurium, rocuronium or analogs and derivatives thereof. The depolarizing agent can be succinylcholine, decamethonium or analogs and derivatives thereof. The muscle relaxant can be a magnesium salt or an organic magnesium compound or analogs and derivatives thereof. The plant extract can be toosendanin, coryneine, banana truck extract, curare, black cohosh, or analogs and derivatives thereof. The amount of black cohosh can be between about 0.01 ml and about 1 ml of black cohosh extract per gram of base cream (about 1 ml of black cohosh extract can be derived from about 2.3 g of dried herb). The polymyxin can be polymyxin B, polymyxin B nonapeptide, polymyxin E, circulin, octapeptin, brevistin, cerexin, polypeptin, stendomycin, deacylpolymyxin, polymyxin octapeptide, polymyxin heptapeptide or analogs and derivatives thereof. The amount of polymyxin B can be between about 800 U/g and about 80,000 U/g. The amount of polymyxin B nonapeptide can be between about 0.04 mg/ml and about 400 mg/ml.

The pharmaceutical composition can further contain a magnesium salt or an organic magnesium compound. The magnesium salt can be magnesium sulfate. The amount of magnesium sulfate can be between about 2 mg/g and about 200 mg/g.

The involuntary facial muscle spasms can be caused by synkinesis, ocular disorders, dystonia, hemifacial spasm, or blepharospasm. Neuropathic pain can be postherpetic neuralgia, diabetic neuropathy, complex regional pain syndrome, spinal cord injury, radiculopathy, migraine headache, myofascial pain, or carpal tunnel syndrome.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a photograph showing the effect of topical cream I on glabellar frown lines

FIG. 2 is a bar graph showing the percentage of subjects with visible wrinkle reduction following treatment with topical cream I.

FIG. 3 is a bar graph showing the reduction in mean values for facial wrinkle scores following treatment with topical cream I.

FIG. 4 is a bar graph showing the percentage of subjects with quantitative improvement of facial wrinkles following treatment with topical cream I.

FIG. 5 is a bar graph showing the best improvement (%) of facial wrinkles in subjects in each parameter following treatment with topical cream I.

FIG. 6 is a photograph showing the effect of topical cream I on glabellar frown lines of a 55 year old female patient.

FIG. 7 is a photograph showing the effect of topical cream I on glabellar frown lines of a 57 year old female patient.

FIG. 8 is a schematic representation illustrating a mouse model for Botox-like compounds and their antagonists.

FIG. 9 is a bar graph showing the effect of topical cream on Botox-induced symptoms in 7 mice.

FIG. 10 is a bar graph showing the effect of topical cream on Botox-induced symptoms in 6 mice.

FIG. 11 is a photograph showing the effect of topical cream III on glabellar frown lines.

FIG. 12 is a photograph showing the effect of topical cream IV on glabellar frown lines.

### DETAILED DESCRIPTION OF THE INVENTION

Disclosed herein are compounds that also have neuromuscular blockade effects, which can be useful in the same clinical settings as botulinum toxin. These compounds may be used topically, providing an advantage over botulinum toxin in application and ease of use. Also disclosed are formulations of topical creams comprising these compounds that are similar to botulinum toxin in their mechanism of action and are effective in both therapeutic and cosmetic settings.

The present invention is based in part on classes of compounds that are functionally similar to botulinum toxin in terms of blocking neuromuscular transmission, but are able to exert physiological effects by topical administration. Additionally, the invention provides pharmaceutical compositions, kits, and methods of localized chemodenervation, methods of treating involuntary facial muscle spasms, methods of reducing facial wrinkles and methods of treating or reducing neuropathic pain using these compounds.

As used herein, the terms “localized chemodenervation” and “localized denervation” mean denervation of a particular anatomical region, usually a muscle or group of anatomically- and/or physiologically-related muscles, which results from administration of a chemodenervating agent, for example a neurotoxin, to the particular anatomical region. This can include a variety of disorders including, but not limited to, involuntary facial muscle spasms, facial wrinkles, and neuropathic pain. As used herein, the terms “neuropathic pain” or “neuropathy pain” mean pain associated with the peripheral and/or central nervous systems.

The term “treating” in its various grammatical forms in relation to the present invention refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease. The term “reducing” in its various grammatical forms in relation to the present invention refers to lessening, curing, reversing, attenuating, alleviating, minimizing, suppressing or halting the deleterious effects of a disease state, disease progression, disease causative agent (e.g., bacteria or viruses) or other abnormal condition.

Any compounds disclosed herein shall also include all derivatives and analogs.

Aminoglycoside Antibiotics

In one aspect, the invention includes aminoglycoside antibiotics or analogs and derivatives thereof used for localized chemodenervation, treatment of involuntary facial muscle spasms, reducing facial wrinkles and treating or reducing neuropathic pain. Aminoglycoside antibiotics are commonly used to treat certain bacterial infections. Preferred aminoglycoside antibiotics include but are not limited to amikacin, gentamicin, kanamycin, neomycin, netilmicin, paromomycin, streptomycin, tobramycin and analogs and derivatives thereof. All of these antibiotics have the similar base chemical structure. As used herein, the term “antibiotic” or “antibiotics” means a chemical substance produced by a microorganism which has the capacity, in dilute solutions, to inhibit the growth of or to kill other microorganisms.

Neuromuscular blockade is a well-known adverse effect that follows the administration of aminoglycoside antibiotics (Corrado et al. Acta Physiol Pharmacol Latinoam 39: 419-430, 1989; Suarez-Kurtz, Acta Physiol Pharmacol Latinoam 39: 407-418, 1989). Many animal studies and clinical case reports have shown that aminoglycoside antibiotics inhibit acetylcholine release at the motor nerve terminal (presynaptic) and/or reducing response to acetylcholine (postsynaptic) (Fiekers, Acta Physiol Pharmacol Ther Latinoam 49: 242-250, 1999).

Aminoglycosides are absorbed very poorly from the gastrointestinal tract. Typical routes of administration are by intramuscular injection, intravenous injection, topical application, or inhalation. Time of peak concentrations occurs between 30 to 120 minutes after intramuscular injection. Intravenous infusion is the preferred administration route for most aminoglycosides. The drugs are diluted in 25 to 100 ml of dextrose or saline, and infused over 30 to 60 minutes.

Tetracyclines

In another aspect, the invention includes tetracyclines and analogs or derivatives thereof used for localized chemodenervation, treatment of involuntary facial muscle spasms and reducing facial wrinkles. Tetracyclines have a modest effect on the release of acetylcholine (Pittinger and Adamson, Annu Rev Pharmacol 12: 169-184, 1972; Wright and Collier, Can J Physiol Pharmacol 54: 926-936, 1976). This effect is partially reversed by calcium, but not by neostigmine. In addition, this effect can be antagonized by 4-aminopyridine, suggesting that its mode of action is on presynaptic calcium ion flux.

Polymyxins

In another aspect, the invention includes polymyxins and derivatives and analogs thereof used for localized chemodenervation, treatment of involuntary facial muscle spasms, reducing facial wrinkles and treating or reducing neuropathic pain. The polymyxins are a group of Bacillus derived peptides of molecular weights of approximately 1200 Daltons. They are basically composed of ten amino acid residues including unusual 2,4-diaminobutyric acid (DAB). The seven residues form a ring structure, with a fatty acid side attached to the N-terminus through an amide linkage. The fatty acid is usually 6-methyloctanoic acid or 6-methylheptanoic acid.

Two major forms of polymyxins are polymyxin B isolated from Bacillus polymyxa and polymyxin E (colistin) from Bacillus colistinus. Structurally, they only differ in one residue at the position 6: D-Phe in polymyxin B and D-Leu in polymyxin E.

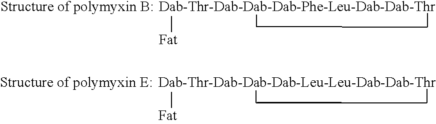

There are many other members of the polymyxin family, with slight structural variations, including but not limited to: circulin, octapeptin, brevistin, cerexin, polypeptin, stendomycin and analogs and derivatives thereof.

Polymyxins, both B and E, are bactericidal to gram-negative bacteria by a dual mechanism of action (Govaerts et al. J Pept Sci 8: 45-55, 2002: Orwa et al. J Chromatogr A 912: 369-373, 2001). Polymyxin first binds to the outer membrane of bacteria. By interacting with lipopolysaccharides in the membrane, polymyxin disrupts and permeabilizes the membrane (sub-lethal action). The second action involves the interaction with the inner or cytoplasmic membrane, resulting the leakage of cytoplasmic components (lethal action).

Approved as topical antibiotic for over 50 years, polymyxin B is also administered orally for GI decontamination prior to abdominal surgery and intrathecally for the treatment of bacterial meningitis. Polymyxin B is widely used as topical antibiotics with the concentration up to 10,000 U per gram (over-the-counter drug).

Polymyxin B produces neuromuscular blockade by acting at pre- and postsynaptic sites (Durant and Lambert, Br J Pharmacol 72: 41-47, 1981). Its postsynaptic effect is believed to be the main cause of the neuromuscular blockade and difficult to reverse by calcium. Of all the antibiotics, polymyxin B is considered to be the most potent neuromuscular blocking agent.

Polymyxin B nonapeptide (PMBN) is derived from the enzymatic removal of the N-terminal Dab residue that is attached to the fatty acid side chain. PMBN is considered to have low anti-bacterial or no anti-bacterial activity at all. Its MIC (minimal inhibitory concentration) for various bacteria increased up to thousand folds compared to its parent molecule, PMB. However, PMBN is still able to interact, like PMB, with bacteria outer membrane and to renders the gram-negative bacteria susceptible to several hydrophobic antibiotics and to the direct bactericidal effect of human serum. A recent study indicated that PMBN greatly increased bacterial killing in the presence of human serum and neutrophils, suggesting its potential use to enhance natural host defense against invading pathogens.

In terms of toxicity, in effective-dose comparisons, PMBN is 15 times less toxic than PMB in an acute-toxicity assay in mice, 25 time less active in releasing histamine from rat mast cells, 100 times less toxic in a eukaryotic cytotoxicity assay, and 150 times less active in causing neuromuscular blockade. In addition, PMBN has a short half-life of approximately 25 minutes in the serum. Generally speaking, PMBN is considered to be significantly far less toxic than PMB—or even nontoxic. However in an unpublished study, Vaara et al claimed that PMBN had a similar nephrotoxicity (proximal tubular injury in young male rats) as PMB.

In addition to PMBN, there are other polymyxin analogs and derivatives, which have similar properties as PMBN, including but not limited to: deacylpolymyxin, polymyxin octapeptide and polymyxin heptapeptide. These analogs and derivatives are generated by removal of the fatty acid side chain, removal of the N-terminal two residues, and removal of the N-terminal three residues, respectively.

The polymyxin can be polymyxin B, polymyxin B nonapeptide, polymyxin E, circulin, octapeptin, brevistin, cerexin, polypeptin, stendomycin, deacylpolymyxin, polymyxin octapeptide, polymyxin heptapeptide or analogs and derivatives thereof. In preferred embodiments, polymyxin is polymyxin B, polymyxin B nonapeptide or analogs and derivatives thereof.

Lincosamides

In another aspect, the invention includes lincosamides or analogs and derivatives thereof used for localized chemodenervation, treatment of involuntary facial muscle spasms, reducing facial wrinkles and treating or reducing neuropathic pain. The pre- and postjunctional effects of the lincosamide antibiotics, clindamycin and lincomycin, were studied by Fiekers et al (J Pharmacol Exp Ther 227: 308-315, 1983) in voltage-clamped transected twitch fibers of costocutaneous muscles of garter snakes (species Thamnophis). Their results suggest that the neuromuscular blocking effects of clindamycin involve both pre-and postjunctional sites, whereas the effects of lincomycin are primarily on the postjunctional receptor-channel complex (Fiekers et al. J Pharmacol Exp Ther 227: 308-315, 1983; Wright and Collier, Can J Physiol Pharmacol 54: 937-944, 1976).

Magnesium

In another aspect, the invention includes magnesium, an organic magnesium compound or analogs and derivatives thereof used for localized chemodenervation, treatment of involuntary facial muscle spasms, reducing facial wrinkles and treating or reducing neuropathic pain. Magnesium is one of the most common cations in the body and plays a fundamental role as a co-factor in more than 300 enzymatic reactions in most metabolisms (Fawcett et al. Br J Anaesth 83: 302-320, 1999). For instance, magnesium is required for the transfer of phosphate groups in signal transduction pathways, ATP involved reactions, replication and transcription of DNA and the translation of mRNA. In addition, magnesium is involved in membrane stability, nerve conduction, iron transport, and calcium-channel activity.

Magnesium deficiency, caused by physiological and dietary conditions, is common and usually multifactorial. Epidemiological studies trace the prevalence of cardiovascular disease and cardiac deaths to the degree of magnesium depletion due to a diet and drinking water low in magnesium.

High magnesium concentration inhibits release acetylcholine from the presynaptic nerve terminal, which can be reversed by calcium. Magnesium sulfate reduces striated muscle contractions and blocks peripheral neuromuscular transmission by reducing acetylcholine release at the myoneural junction. In emergency care, magnesium sulfate is used in the management of seizures associated with toxemia of pregnancy and in uterine relaxation (to inhibit contractions of premature labor).

Many different magnesium salts are used in clinical setting, including magnesium oxide, mineral magnesium salts (e.g., sulfate, nitrate, chloride and carbonate) and organic magnesium (e.g., ascorbate, acetate, bicitrate, methionate, levulinate, glycero-phosphate, gluconate, aspartate, propionate, lactate, fumarate, glutamate and pyrrolidone-carboxylate).

Plant and Herb Extracts

In another aspect, the invention includes plant and herb extracts or analogs and derivatives thereof used for localized chemodenervation, treatment of involuntary facial muscle spasms, reducing facial wrinkles and treating or reducing neuropathic pain. As used herein, the term “plant extract” means compounds derived from plants, herbs or botanicals. Preferred plant extracts/botanicals include but are not limited to toosendanin, coryneine, banana truck extract, curare, black cohosh (ciminifuga racemosa) or analogs and derivatives thereof.

Toosendanin is a triterpenoid derivative of molecular weight of 574 (Ding et al. Neurosci Res 41: 243-249, 2001; Wang and Shi, Neurosci Res 40: 211-215, 2001; Xu and Shi, Brain Res 631: 46-50, 1993). As an active compound in Fructus Maliae Toosendan or Cortex Maliae, and in Chinese traditional medicine, toosendanin is reported to be a neuromuscular blocker acting on the presynaptic terminals. The blocking action of toosendanin is preceded by a facilitating phase, which may last for several days. It is interesting that during such a facilitating phase, the tolerance of the neuromuscular junction towards botulinum toxin is enhanced significantly.

Aconite root is commonly used in Chinese and Japanese herbal medicine for relieving muscle pain. Isolated from aconite roots, coryneine is a quaternary ammonium derivative of dopamine, which exhibits a depolarizing neuromuscular blocking action (Nojima et al. J Asian Nat Prod Res 2: 195-203, 2000). Kimura et al showed that coryneine at 20-150 uM blocked the nerve-evoked twitch response, which was reversed by neostigmine, a cholinesterase inhibitor (Kimura et al. Biol Pharm Bull 18: 691-695, 1995).

It was reported that the juice of the banana truck induces a muscle paralyzing effect in both directly and indirectly stimulated preparations (Singh et al. Arch Int Pharmacodyn Ther 324: 105-113, 1993; Singh and Dryden, Toxicon 23: 973-981, 1985). The neuromuscular blockade was reversed by calcium, but only when added before complete paralysis of the muscle. A chemical composition study of banana truck revealed that the active component responsible for muscle paralysis mainly consisted of monopotassium ovalate. However, another study claimed that two major active principles causing muscle paralysis are potassium nitrate and magnesium nitrate.

An extract from the bark and stems of curare (Chondodendron tomentosum) is the source of a potent isoquinoline alkaloid used in the deadly poison curare. Amazonian Indians use the gummy extract to coat the poison darts of their blowguns. The alkaloid D-tubocurarine blocks acetylcholine receptor sites at neuromuscular junctions, causing relaxation and paralysis of muscles, including respiratory organs and the heart (Mallart and Molgo, J Physiol 276: 343-352, 1978; Manalis, Nature 267: 366-368, 1977). D-tubocurarine has been used to relax the heart muscle during open-heart surgery. It has also been used to treat the spastic paralysis of tetanus toxin from the bacterium Clostridium botulinum.

Black cohosh (ciminifuga racemosa) from the family Ranuncleaceae is a perennial plant with a smooth, furrowed stem, cohosh grows from 3-9 feet in height. Its leaves are ternate, then pinnate, and are irregularly-shaped. There are 2-5 leaflets, egg-shaped or oblong and irregularly toothed and cut. Small white or yellow flowers grow in long racemes from June to August. The rhizome and root is often dried and collected in autumn and used medicinally.

Black cohosh was introduced to American medicine by native Indians, who called it “squaw root” in reference to its common use: treatment of uterine disorders. Native American women made a tea from the plant's resinous roots and rhizome to soothe menstrual cramps and promote menstruation. Black cohosh was also important in folk medicine as a childbirth aid that eased delivery by stimulating the uterus. Native Americans and American colonists also valued it as a treatment for rheumatism and sore throats. As early as 1787, the plant attracted the interest of Europeans. Eclectic physicians, a nineteenth-century branch of early American medicine, made black cohosh one of their central healing botanicals for women. From 1820 to 1926, the herb was listed as an official drug in the United States Pharmacopoeia. Like many phytomedicines, however, it eventually fell out of favor with the American medical community. It continued to be used in Europe, and today, in Germany, it is a government-approved treatment for premenstrual syndrome, painful menstruation, and nervous conditions connected to menopause.

Clinical studies indicate black cohosh promotes and/or restores healthy menstrual activity; soothes irritation and congestion of the uterus, cervix and vagina; relieves the pain and distress of pregnancy; contributes to quick, easy, and uncomplicated deliveries; and promotes uterine involution and recovery. Other studies have demonstrated that black cohosh extract contains substances that reduce luteinizing hormone (LH). LH is associated with menopausal symptoms (e.g., hot flashes, night sweats, nervousness, irritability, sleeplessness and depressive moods). Studies suggest black cohosh extract is capable of providing relief of menopausal symptoms comparable to that of hormone-replacement therapy. One study found black cohosh extract similar to estrogen in reducing hot flashes, headache and joint pain. Another study found black cohosh extract superior to placebo in relieving hot flashes and reducing vaginal dryness.

Black cohosh is considered to have several therapeutic effects, it displays hypotensive, vasodilatory, estrogenic, anti-inflammatory, and uterine contractile activity. Although the exact mode of action remains unresolved, the herb appears to act both directly on the tissues of the reproductive system, and indirectly through the nervous system. Black cohosh extract did not stimulate the growth of estrogen receptor positive breast cancer cells. Black cohosh's general hypotensive property is most likely due to the presence of an active, water insoluable resin. It is hypothesized the active principle of the resin influences the central circulation directly as well as indirectly through inhibition of vasomotor centers. Black cohosh's anti-spasmodic properties prevent or ease spasms or cramps in the muscles of the body. It may function as a primary nerve and smooth muscle relaxant.

Black cohosh is now most commonly used to relieve the symptoms commonly associated with menopause such as hot flashes, night sweats, and menstrual cramps. Black cohosh is also noted for its ability to alleviate the symptoms of PMS. Harvard Women's health Watch called it “the woman's herb”. There is also some evidence to suggest it helps alleviate depression, and that it harbors anti-inflammatory properties; some people, therefore, use it to mitigate muscle pains and aches. Other benefits include treating coughs, due to its ability to clear out bodily mucus, and as a treatment for tinnitus, a condition in which the ears exhibit a chronic ringing.

Black cohosh is most commonly taken by oral administration as diet supplement. Currently, there are two topical creams that contain black cohosh: Nature's Balance Cream contains premium wild yam root, along with black cohosh, to help women maintain hormonal balance. Horizon progesterone cream contains progesterone from soy, Mexican wild yam and black cohosh root extracts for claims like hormone balance, anti-depressant, sex drive and cancer prevention.

A recent report (15 Jul. 2003, Dow Jones & Reuters) confirmed the safety of black cohosh based on most comprehensive scientific review to date. The most commonly reported side effects are mild gastric complaints. High doses may cause headaches, vomiting, and dizziness. There have been no documented herb-drug interactions. Black cohosh is not recommended for those who are pregnant or breast feeding because of a lack of data. Its use is not recommended for more than 6 months because of a lack of long-term data.

For menopausal women, black cohosh (RemiFemin) can safely be taken continuously, twice a day (40 mg), for six months at a time. After six months, women should reassess their symptoms and treatment needs as symptoms typically fluctuate throughout the multi-year menopausal process. RemiFemin Menopause is the prominent OB/GYN-recommended over-the-counter menopausal therapy. No significant drug interactions have been reported for RemiFemin in its over 40 years of worldwide use and adverse events have been limited to mild, temporary upset stomach.

Other plants or herbs that have demonstrated neuromuscular blocking activity include, but are not limited to, piper methysticum (Singh, J Ethnopharmacol 7: 267-276, 1983), Atractylodes lancea (Kimura et al. Neuropharmacology 30: 835-841, 1991), passiflora incarnata, camellia sinensis (Das et al. J Ethnopharmacol 57: 197-201, 1997), sarcolobus globosus (Mustafa, Toxicon 31: 67-74, 1993), ipomoea fistulosa (Abdelhadi et al. Clin Exp Pharmacol Physiol 13: 169-171, 1986), piper sarmentosum (Ridtitid et al. J Ethnopharmacol 61: 135-142, 1998), Saccharum officinarum and Psidium guajava (Re et al. Pharmacol Res 39: 239-245, 1999).

Muscle Relaxants

In another aspect, the invention includes muscle relaxants or analogs and derivatives thereof used for localized chemodenervation, treatment of involuntary facial muscle spasms, reducing facial wrinkles and treating or reducing neuropathic pain. Studies of nerve-impulse transmission at neuromuscular junctions have indicated that acetylcholine released from the presynaptic site binds to specific receptors on postsynaptic membrane of motor end plate. Such a binding produces a large change in ion permeability properties (depolarizing effect) of the postsynaptic membrane.

The transmission of acetylcholine at the neuromuscular joints can be blocked by either non-depolarizing agents or depolarizing agents. The non-depolarizing agents compete with acetylcholine for the postsynaptic receptor site, thus preventing depolarization. Preferred non-depolarizing agents include, but are not limited to, pancuronium, vecuronium, mivacurium, rocuronium or analogs and derivatives thereof. The depolarizing blocking agents act like an excess of acetylcholine to depolarize the muscle receptor site and prevent its repolarization. Thus, there is an initial depolarization resulting in muscle contraction. Since the muscle receptor site cannot depolarize, complete skeletal muscle relaxation follows. Preferred depolarizing agents include, but are not limited to, succinylcholine, decamethonium or analogs and derivatives thereof.

Most of those muscle relaxants described above offer rapid onset (several minutes) and short duration (up to two hours), and have been extensively used as anesthesia in surgical procedures.

Although the neuromuscular blocking properties of the above said compounds have been reported in the literature, their clinical utilities have not been fully explored, except for the muscle relaxants and magnesium used as anesthesia in surgical procedures and other medical conditions. In fact, neuromuscular blockade is considered as the undesirable side effect for the antibiotics mentioned above. Furthermore, none of the above said compounds, with exception for polymyxin B, has been topically used in clinical settings.

Methods for Localized Chemodenervation

Aminoglycoside Antibiotics:

The present invention includes methods for localized chemodenervation. In one aspect, the present invention includes a method for localized chemodenervation by topically administering a pharmaceutical composition comprising a pharmaceutically effective amount of an antibiotic and a pharmaceutically acceptable carrier.

In a preferred embodiment, the method described above comprises a pharmaceutically effective amount of aminoglycoside antibiotic or analogs and derivatives thereof and a pharmaceutically acceptable carrier. The aminoglycoside antibiotic can be amikacin, gentamicin, kanamycin, neomycin, netilmicin, paromomycin, streptomycin, tobramycin or analogs and derivatives thereof. The pharmaceutical composition can further comprise a magnesium salt or an organic magnesium compound.

In another preferred embodiment, the pharmaceutical composition for topical administration for localized chemodenervation comprises effective amounts of an aminoglycoside antibiotic or analogs and derivatives thereof and a magnesium salt or organic magnesium compound in a medium compatible with human skin. The aminoglycoside antibiotic can be neomycin and the magnesium salt can be magnesium sulfate. In preferred embodiments, the amount of neomycin is between about 2.5 mg/g and 250 mg/g and the amount of magnesium sulfate is between 2 mg/g and 200 mg/g. In a more preferred embodiment, the amount of neomycin can be about 125 mg/g and the amount of magnesium sulfate can be about 100 mg/g. The most preferred embodiment is a pharmaceutical composition comprising Cream II (described below).

Tetracyclines:

The invention also includes a method for localized chemodenervation by topically administering a pharmaceutical composition for comprising a pharmaceutically effective amount of tetracycline or analogs and derivatives thereof and a pharmaceutically acceptable carrier. In a preferred embodiment, the pharmaceutical composition can further comprise a magnesium salt or an organic magnesium compound.

Polymyxin:

In another aspect, the invention includes a method for localized chemodenervation by topically administering a pharmaceutical composition comprising a pharmaceutically effective amount of a polymyxin or analogs and derivatives thereof and a pharmaceutically acceptable carrier. In preferred embodiments, polymyxin is polymyxin B, polymyxin B nonapeptide or analogs and derivatives thereof. In a further preferred embodiment, the pharmaceutical composition can further comprise a magnesium salt or an organic magnesium compound.

In another preferred aspect, the pharmaceutical composition for topical administration for localized chemodenervation comprises effective amounts of a polymyxin or analogs and derivatives thereof and a magnesium salt or organic magnesium compound in a medium compatible with human skin. The magnesium salt can be magnesium sulfate. In preferred embodiments, polymyxin is polymyxin B, polymyxin B nonapeptide or analogs and derivatives thereof. In further preferred embodiments, the amount of polymyxin B is between about 800 U/g and about 80,000 U/g and the amount of magnesium sulfate is between 2 mg/g and 200 mg/g. In a more preferred embodiment, the amount of polymyxin B is between about 4,000 U/g and about 40,000 U/g and the amount of magnesium sulfate is about 100 mg/g. In another embodiment, the amount of polymyxin B nonapeptide can be between about 0.04 mg/ml and about 400 mg/ml and the amount of magnesium sulfate is between 2 mg/g and 200 mg/g. In a preferred embodiment, the amount of polymyxin B nonapeptide can be between about 0.4 mg/ml and about 40 mg/ml and the amount of magnesium sulfate is about 100 mg/g. The most preferred embodiment is a pharmaceutical composition comprising Cream I and a pharmaceutical composition comprising Cream m (described below).

Lincosamides:

The invention also includes a method for localized chemodenervation by topically administering a pharmaceutical composition comprising a pharmaceutically effective amount of lincosamide or analogs and derivatives thereof and a pharmaceutically acceptable carrier. The lincosamide can be clindamycin, lincomycin or analogs and derivatives thereof. In a preferred embodiment, the pharmaceutical composition can further comprise a magnesium salt or an organic magnesium compound.

Muscle Relaxants:

In another aspect, the invention includes a method for localized chemodenervation by topically administering a pharmaceutical composition comprising a pharmaceutically effective amount of a muscle relaxant or analogs and derivatives thereof and pharmaceutically acceptable carrier. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt or an organic magnesium compound.

Plant/Herb Extracts:

The invention also includes a method for localized chemodenervation by topically administering a pharmaceutical composition comprising a pharmaceutically effective amount of one or more compounds selected from the group consisting of a magnesium salt, an organic magnesium compound, a plant extract, or analogs and derivatives thereof and a pharmaceutically acceptable carrier.

In a preferred embodiment, the method described above comprises a pharmaceutically effective amount of plant extract or analogs and derivatives thereof and a pharmaceutically acceptable carrier. The plant extract can be toosendanin, coryneine, banana truck extract, curare, black cohosh or analogs and derivatives thereof. The pharmaceutical composition can further comprise a magnesium salt or an organic magnesium compound.

In another preferred embodiment, the pharmaceutical composition for topical administration for localized chemodenervation comprises effective amounts of plant extract or analogs and derivatives thereof and a magnesium salt or organic magnesium compound in a medium compatible with human skin. The plant extract can be black cohosh and the magnesium salt can be magnesium sulfate. In preferred embodiments, the amount of plant extract is between about 0.01 ml and about 1 ml of plant extract per gram of base cream and the amount of magnesium sulfate is between 2 mg/g and 200 mg/g (about 1 ml of plant extract (i.e. black cohosh) can be derived from about 2.3 g of dried herb). In a more preferred embodiment, the amount of plant extract is between about 0.01 ml and about 1 ml of plant extract per gram of base cream and the amount of magnesium sulfate can be about 100 mg/g (about 1 ml of plant extract (i.e. black cohosh) can be derived from about 2.3 g of dried herb). The most preferred embodiment is a pharmaceutical composition comprising Cream IV (described below).

Methods for Treating Involuntary Facial Muscle Spasms

Aminoglycoside Antibiotics:

The present invention includes methods for treating involuntary facial muscle spasms. The involuntary facial muscle spasms can be caused by synkinesis, ocular disorders, dystonia, hemifacial spasm, or blepharospasm. In one aspect, the present invention includes a method for treating involuntary facial muscle spasms by topically administering a pharmaceutical composition comprising a pharmaceutically effective amount of an antibiotic and a pharmaceutically acceptable carrier.

In a preferred embodiment, the method described above comprises a pharmaceutically effective amount of aminoglycoside antibiotic or analogs and derivatives thereof and a pharmaceutically acceptable carrier. The aminoglycoside antibiotic can be amikacin, gentamicin, kanamycin, neomycin, netilmicin, paromomycin, streptomycin, tobramycin or analogs and derivatives thereof. The pharmaceutical composition can further comprise a magnesium salt or an organic magnesium compound.

In another preferred embodiment, the pharmaceutical composition for topical administration for treating involuntary facial muscle spasms comprises effective amounts of an aminoglycoside antibiotic or analogs and derivatives thereof and a magnesium salt or organic magnesium compound in a medium compatible with human skin. The aminoglycoside antibiotic can be neomycin and the magnesium salt can be magnesium sulfate. In preferred embodiments, the amount of neomycin is between about 2.5 mg/g and 250 mg/g and the amount of magnesium sulfate is between 2 mg/g and 200 mg/g. In a more preferred embodiment, the amount of neomycin can be about 125 mg/g and the amount of magnesium sulfate can be about 100 mg/g. The most preferred embodiment is a pharmaceutical composition comprising Cream II (described below).

Tetracyclines:

The invention also includes a method for treating involuntary facial muscle spasms by topically administering a pharmaceutical composition for comprising a pharmaceutically effective amount of tetracycline or analogs and derivatives thereof and a pharmaceutically acceptable carrier. In a preferred embodiment, the pharmaceutical composition can further comprise a magnesium salt or an organic magnesium compound.

Polymyxin:

In another aspect, the invention includes a method for treating involuntary facial muscle spasms by topically administering a pharmaceutical composition comprising a pharmaceutically effective amount of a polymyxin or analogs and derivatives thereof and a pharmaceutically acceptable carrier. In preferred embodiments, polymyxin is polymyxin B, polymyxin B nonapeptide or analogs and derivatives thereof. In a further preferred embodiment, the pharmaceutical composition can further comprise a magnesium salt or an organic magnesium compound.

In another preferred aspect, the pharmaceutical composition for topical administration for treating involuntary facial muscle spasms comprises effective amounts of a polymyxin or analogs and derivatives thereof and a magnesium salt or organic magnesium compound in a medium compatible with human skin. The magnesium salt can be magnesium sulfate. In preferred embodiments, polymyxin is polymyxin B, polymyxin B nonapeptide or analogs and derivatives thereof. In further preferred embodiments, the amount of polymyxin B is between about 800 U/g and about 80,000 U/g and the amount of magnesium sulfate is between 2 mg/g and 200 mg/g. In a more preferred embodiment, the amount of polymyxin B is between about 4,000 U/g and about 40,000 U/g and the amount of magnesium sulfate is about 100 mg/g. In another embodiment, the amount of polymyxin. B nonapeptide can be between about 0.04 mg/ml and about 400 mg/ml and the amount of magnesium sulfate is between 2 mg/g and 200 mg/g. In a preferred embodiment, the amount of polymyxin B nonapeptide can be between about 0.4 mg/ml and about 40 mg/ml and the amount of magnesium sulfate is about 100 mg/g. The most preferred embodiment is a pharmaceutical composition comprising Cream I and a pharmaceutical composition comprising Cream III (described below).

Lincosamides:

The invention also includes a method for treating involuntary facial muscle spasms by topically administering a pharmaceutical composition comprising a pharmaceutically effective amount of lincosamide or analogs and derivatives thereof and a pharmaceutically acceptable carrier. The lincosamide can be clindamycin, lincomycin or analogs and derivatives thereof. In a preferred embodiment, the pharmaceutical composition can further comprise a magnesium salt or an organic magnesium compound.

Muscle Relaxants:

In another aspect, the invention includes a method for treating involuntary facial muscle spasms by topically administering a pharmaceutical composition comprising a pharmaceutically effective amount of a muscle relaxant or analogs and derivatives thereof and pharmaceutically acceptable carrier. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt or an organic magnesium compound.

Plant/Herb Extracts:

The invention also includes a method for treating involuntary facial muscle spasms by topically administering a pharmaceutical composition comprising a pharmaceutically effective amount of one or more compounds selected from the group consisting of a magnesium salt, an organic magnesium compound, a plant extract, or analogs and derivatives thereof and a pharmaceutically acceptable carrier.

In a preferred embodiment, the method described above comprises a pharmaceutically effective amount of plant extract or analogs and derivatives thereof and a pharmaceutically acceptable carrier. The plant extract can be toosendanin, coryneine, banana truck extract, curare, black cohosh or analogs and derivatives thereof. The pharmaceutical composition can further comprise a magnesium salt or an organic magnesium compound.

In another preferred embodiment, the pharmaceutical composition for topical administration for treating involuntary facial muscle spasms comprises effective amounts of plant extract or analogs and derivatives thereof and a magnesium salt or organic magnesium compound in a medium compatible with human skin. The plant extract can be black cohosh and the magnesium salt can be magnesium sulfate. In preferred embodiments, the amount of plant extract is between about 0.01 ml and about 1 ml of plant extract per gram of base cream and the amount of magnesium sulfate is between 2 mg/g and 200 mg/g (about 1 ml of plant extract (i.e. black cohosh) can be derived from about 2.3 g of dried herb). In a more preferred embodiment, the amount of plant extract is between about 0.01 ml and about 1 ml of plant extract per gram of base cream and the amount of magnesium sulfate can be about 100 mg/g (about 1 ml of plant extract (i.e. black cohosh) can be derived from about 2.3 g of dried herb). The most preferred embodiment is a pharmaceutical composition comprising Cream IV (described below).

Methods for Reducing Facial Wrinkles

Aminoglycoside Antibiotics:

The present invention includes methods for reducing facial wrinkles. In one aspect, the present invention includes a method for reducing facial wrinkles by topically administering a pharmaceutical composition comprising a pharmaceutically effective amount of an antibiotic and a pharmaceutically acceptable carrier.

In a preferred embodiment, the method described above comprises a pharmaceutically effective amount of aminoglycoside antibiotic or analogs and derivatives thereof and a pharmaceutically acceptable carrier. The aminoglycoside antibiotic can be amikacin, gentamicin, kanamycin, neomycin, netilmicin, paromomycin, streptomycin, tobramycin or analogs and derivatives thereof. The pharmaceutical composition can further comprise a magnesium salt or an organic magnesium compound.

In another preferred embodiment, the pharmaceutical composition for topical administration for reducing facial wrinkles comprises effective amounts of an aminoglycoside antibiotic or analogs and derivatives thereof and a magnesium salt or organic magnesium compound in a medium compatible with human skin. The aminoglycoside antibiotic can be neomycin and the magnesium salt can be magnesium sulfate. In preferred embodiments, the amount of neomycin is between about 2.5 mg/g and 250 mg/g and the amount of magnesium sulfate is between 2 mg/g and 200 mg/g. In a more preferred embodiment, the amount of neomycin can be about 125 mg/g and the amount of magnesium sulfate can be about 100 mg/g. The most preferred embodiment is a pharmaceutical composition comprising Cream II (described below).

Tetracyclines:

The invention also includes a method for reducing facial wrinkles by topically administering a pharmaceutical composition for comprising a pharmaceutically effective amount of tetracycline or analogs and derivatives thereof and a pharmaceutically acceptable carrier. In a preferred embodiment, the pharmaceutical composition can further comprise a magnesium salt or an organic magnesium compound.

Polymyxin:

In another aspect, the invention includes a method for reducing facial wrinkles by topically administering a pharmaceutical composition comprising a pharmaceutically effective amount of a polymyxin or analogs and derivatives thereof and a pharmaceutically acceptable carrier. In preferred embodiments, polymyxin is polymyxin B, polymyxin B nonapeptide or analogs and derivatives thereof. In a further preferred embodiment, the pharmaceutical composition can further comprise a magnesium salt or an organic magnesium compound.

In another preferred aspect, the pharmaceutical composition for topical administration for reducing facial wrinkles comprises effective amounts of a polymyxin or analogs and derivatives thereof and a magnesium salt or organic magnesium compound in a medium compatible with human skin. The magnesium salt can be magnesium sulfate. In preferred embodiments, polymyxin is polymyxin B, polymyxin B nonapeptide or analogs and derivatives thereof. In further preferred embodiments, the amount of polymyxin B is between about 800 U/g and about 80,000 U/g and the amount of magnesium sulfate is between 2 mg/g and 200 mg/g. In a more preferred embodiment, the amount of polymyxin B is between about 4,000 U/g and about 40,000 U/g and the amount of magnesium sulfate is about 100 mg/g. In another embodiment, the amount of polymyxin B nonapeptide can be between about 0.04 mg/ml and about 400 mg/ml and the amount of magnesium sulfate is between 2 mg/g and 200 mg/g. In a preferred embodiment, the amount of polymyxin B nonapeptide can be between about 0.4 mg/ml and about 40 mg/ml and the amount of magnesium sulfate is about 100 mg/g. The most preferred embodiment is a pharmaceutical composition comprising Cream I and a pharmaceutical composition comprising Cream m (described below).

Lincosamides:

The invention also includes a method for reducing facial wrinkles by topically administering a pharmaceutical composition comprising a pharmaceutically effective amount of lincosamide or analogs and derivatives thereof and a pharmaceutically acceptable carrier. The lincosamide can be clindamycin, lincomycin or analogs and derivatives thereof. In a preferred embodiment, the pharmaceutical composition can further comprise a magnesium salt or an organic magnesium compound.

Muscle Relaxants:

In another aspect, the invention includes a method for reducing facial wrinkles by topically administering a pharmaceutical composition comprising a pharmaceutically effective amount of a muscle relaxant or analogs and derivatives thereof and pharmaceutically acceptable carrier. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt or an organic magnesium compound.

Plant/Herb Extracts:

The invention also includes a method for reducing facial wrinkles by topically administering a pharmaceutical composition comprising a pharmaceutically effective amount of one or more compounds selected from the group consisting of a magnesium salt, an organic magnesium compound, a plant extract, or analogs and derivatives thereof and a pharmaceutically acceptable carrier.

In a preferred embodiment, the method described above comprises a pharmaceutically effective amount of plant extract or analogs and derivatives thereof and a pharmaceutically acceptable carrier. The plant extract can be toosendanin, coryneine, banana truck extract, curare, black cohosh or analogs and derivatives thereof. The pharmaceutical composition can further comprise a magnesium salt or an organic magnesium compound.

In another preferred embodiment, the pharmaceutical composition for topical administration for reducing facial wrinkles comprises effective amounts of plant extract or analogs and derivatives thereof and a magnesium salt or organic magnesium compound in a medium compatible with human skin. The plant extract can be black cohosh and the magnesium salt can be magnesium sulfate. In preferred embodiments, the amount of plant extract is between about 0.01 ml and about 1 ml of plant extract per gram of base cream and the amount of magnesium sulfate is between 2 mg/g and 200 mg/g (about 1 ml of plant extract (i.e. black cohosh) can be derived from about 2.3 g of dried herb). In a more preferred embodiment, the amount of plant extract is between about 0.01 ml and about 1 ml of plant extract per gram of base cream and the amount of magnesium sulfate can be about 100 mg/g (about 1 ml of plant extract (i.e. black cohosh) can be derived from about 2.3 g of dried herb). The most preferred embodiment is a pharmaceutical composition comprising Cream IV (described below).

Methods for Treating or Reducing Neuropathic Pain

Aminoglycoside Antibiotics:

The present invention includes methods for treating or reducing neuropathic pain. Neuropathic pain can be postherpetic neuralgia, diabetic neuropathy, complex regional pain syndrome, spinal cord injury, radiculopathy, migraine headache, myofascial pain, or carpal tunnel syndrome. In one aspect, the present invention includes a method for treating or reducing neuropathic pain by topically administering a pharmaceutical composition comprising a pharmaceutically effective amount of an antibiotic and a pharmaceutically acceptable carrier.

In a preferred embodiment, the method described above comprises a pharmaceutically effective amount of aminoglycoside antibiotic or analogs and derivatives thereof and a pharmaceutically acceptable carrier. The aminoglycoside antibiotic can be amikacin, gentamicin, kanamycin, neomycin, netilmicin, paromomycin, streptomycin, tobramycin or analogs and derivatives thereof. The pharmaceutical composition can further comprise a magnesium salt or an organic magnesium compound.

In another preferred embodiment, the pharmaceutical composition for topical administration for treating or reducing neuropathic pain comprises effective amounts of an aminoglycoside antibiotic or analogs and derivatives thereof and a magnesium salt or organic magnesium compound in a medium compatible with human skin. The aminoglycoside antibiotic can be neomycin and the magnesium salt can be magnesium sulfate. In preferred embodiments, the amount of neomycin is between about 2.5 mg/g and 250 mg/g and the amount of magnesium sulfate is between 2 mg/g and 200 mg/g. In a more preferred embodiment, the amount of neomycin can be about 125 mg/g and the amount of magnesium sulfate can be about 100 mg/g. The most preferred embodiment is a pharmaceutical composition comprising Cream II (described below).

Tetracyclines:

The invention also includes a method for treating or reducing neuropathic pain by topically administering a pharmaceutical composition for comprising a pharmaceutically effective amount of tetracycline or analogs and derivatives thereof and a pharmaceutically acceptable carrier. In a preferred embodiment, the pharmaceutical composition can further comprise a magnesium salt or an organic magnesium compound.

Polymyxin:

In another aspect, the invention includes a method for treating or reducing neuropathic pain by topically administering a pharmaceutical composition comprising a pharmaceutically effective amount of a polymyxin or analogs and derivatives thereof and a pharmaceutically acceptable carrier. In preferred embodiments, polymyxin is polymyxin B, polymyxin B nonapeptide or analogs and derivatives thereof. In a further preferred embodiment, the pharmaceutical composition can further comprise a magnesium salt or an organic magnesium compound.

In another preferred aspect, the pharmaceutical composition for topical administration for treating or reducing neuropathic pain comprises effective amounts of a polymyxin or analogs and derivatives thereof and a magnesium salt or organic magnesium compound in a medium compatible with human skin. The magnesium salt can be magnesium sulfate. In preferred embodiments, polymyxin is polymyxin B, polymyxin B nonapeptide or analogs and derivatives thereof. In further preferred embodiments, the amount of polymyxin B is between about 800 U/g and about 80,000 U/g and the amount of magnesium sulfate is between 2 mg/g and 200 mg/g. In a more preferred embodiment, the amount of polymyxin B is between about 4,000 U/g and about 40,000 U/g and the amount of magnesium sulfate is about 100 mg/g. In another embodiment, the amount of polymyxin B nonapeptide can be between about 0.04 mg/ml and about 400 mg/ml and the amount of magnesium sulfate is between 2 mg/g and 200 mg/g. In a preferred embodiment, the amount of polymyxin B nonapeptide can be between about 0.4 mg/ml and about 40 mg/ml and the amount of magnesium sulfate is about 100 mg/g. The most preferred embodiment is a pharmaceutical composition comprising Cream I and a pharmaceutical composition comprising Cream III (described below).

Lincosamides:

The invention also includes a method for treating or reducing neuropathic pain by topically administering a pharmaceutical composition comprising a pharmaceutically effective amount of lincosamide or analogs and derivatives thereof and a pharmaceutically acceptable carrier. The lincosamide can be clindamycin, lincomycin or analogs and derivatives thereof. In a preferred embodiment, the pharmaceutical composition can further comprise a magnesium salt or an organic magnesium compound.

Muscle Relaxants:

In another aspect, the invention includes a method for treating or reducing neuropathic pain by topically administering a pharmaceutical composition comprising a pharmaceutically effective amount of a muscle relaxant or analogs and derivatives thereof and pharmaceutically acceptable carrier. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt or an organic magnesium compound.

Plant/Herb Extracts:

The invention also includes a method for treating or reducing neuropathic pain by topically administering a pharmaceutical composition comprising a pharmaceutically effective amount of one or more compounds selected from the group consisting of a magnesium salt, an organic magnesium compound, a plant extract, or analogs and derivatives thereof and a pharmaceutically acceptable carrier.

In a preferred embodiment, the method described above comprises a pharmaceutically effective amount of plant extract or analogs and derivatives thereof and a pharmaceutically acceptable carrier. The plant extract can be toosendanin, coryneine, banana truck extract, curare, black cohosh or analogs and derivatives thereof. The pharmaceutical composition can further comprise a magnesium salt or an organic magnesium compound.

In another preferred embodiment, the pharmaceutical composition for topical administration for treating or reducing neuropathic pain comprises effective amounts of plant extract or analogs and derivatives thereof and a magnesium salt or organic magnesium compound in a medium compatible with human skin. The plant extract can be black cohosh and the magnesium salt can be magnesium sulfate. In preferred embodiments, the amount of plant extract is between about 0.01 ml and about 1 ml of plant extract per gram of base cream and the amount of magnesium sulfate is between 2 mg/g and 200 mg/g (about 1 ml of plant extract (i.e. black cohosh) can be derived from about 2.3 g of dried herb). In a more preferred embodiment, the amount of plant extract is between about 0.01 ml and about 1 ml of plant extract per gram of base cream and the amount of magnesium sulfate can be about 100 mg/g (about 1 ml of plant extract (i.e. black cohosh) can be derived from about 2.3 g of dried herb). The most preferred embodiment is a pharmaceutical composition comprising Cream IV (described below).

Pharmaceutical Compositions

The compounds, e.g., antibiotics (including but not limited to: aminoglycoside antibiotics, tetracyclines, polymyxin (including but not limited to polymyxin B and polymyxin B nonapeptide), and lincosamides), magnesium and muscle relaxants (also referred to herein as “active compounds”) of the invention, and derivatives, fragments, analogs and homologs thereof, can be incorporated into pharmaceutical compositions suitable for administration. Such compositions typically comprise the active compounds and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Transdermal or Transmucosal Administration:

Systemic administration can also be by transmucosal or transdermal means. In a preferred embodiment, the pharmaceutical compositions of the present invention are administered topically. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment of the present invention, for topical application, the compositions of the invention comprise a medium (vehicle, diluent or carrier), which is compatible with human skin. These compositions can be, in particular, in the form of aqueous, alcoholic or aqueous/alcoholic solutions, ointments, lotions, gels, water-in-oil or oil-in-water emulsions having the appearance of a cream or a gel, microemulsions or aerosols, or alternatively in the form of vesicular dispersions containing ionic and/or nonionic lipids. These pharmaceutical dosage units are formulated according to the usual techniques in the fields under consideration.

In known fashion, the compositions of the invention can also contain adjuvants and additives that are common in the corresponding fields, such as hydrophilic or lipophilic gelling agents, preservatives, antioxidants, solvents, fragrances, fillers, UV-screening agents and dyestuffs and colorants. Moreover, these compositions can contain hydrophilic or lipophilic active agents. The amounts of these various adjuvants, additives or active agents are those used conventionally in the cosmetic or pharmaceutical field, and, for example, constitute from 0.01% to 5% of the total weight of the composition. Depending on their nature, these adjuvants or active agents can be introduced into the fatty phase, into the aqueous phase and/or into lipid vesicles.

In order to deliver the said compounds to the deep layers of the skin, the mixture can also be incorporated into the aqueous chambers of liposomes commercially prepared from phosphatidylcholine (lecithin). Liposomes are used widely because of their ability of penetrating deeply into the skin. In addition, applying controlled-release delivery technology known to the industry, function ingredients can be delivered at a pre-determined rate to achieve long-lasting therapeutic effects.

It is especially advantageous to formulate topically administered pharmaceutical compositions in a dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. The appropriate dosage or dose is often referred to as an effective amount. As used herein, the terms “effective amount” and “pharmaceutically effective amount” mean the ability to denervate or treat or reduce a disease or a disorder or a symptom thereof or to the amount of a compound to produce the desired effect in a subject or cell. An effective amount of a pharmaceutical agent is that which provides an objectively identifiable improvement as noted by the clinician or other qualified observer.

Polymyxin Pharmaceutical Compositions:

In one aspect, the invention includes a pharmaceutical composition for topical administration for localized chemodenervation comprising pharmaceutically effective amounts of polymyxin or analogs and derivatives thereof and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of polymyxin or analogs and derivatives thereof and a magnesium salt or organic magnesium compound in a pharmaceutically acceptable medium compatible with human skin. In a further aspect, the invention includes a pharmaceutical composition for topical administration for reducing facial wrinkles comprising pharmaceutically effective amounts of polymyxin or analogs and derivatives thereof and a magnesium salt or organic magnesium compound in a pharmaceutically acceptable medium compatible with human skin. In yet a further aspect, the invention includes a pharmaceutical composition for topical administration for treating or reducing neuropathic pain comprising pharmaceutically effective amounts of polymyxin or analogs and derivatives thereof and a magnesium salt or organic magnesium compound in a pharmaceutically acceptable medium compatible with human skin.

The magnesium salt can be magnesium sulfate. The polymyxin can be polymyxin B, polymyxin B nonapeptide, polymyxin E, circulin, octapeptin, brevistin, cerexin, polypeptin, stendomycin, deacylpolymyxin, polymyxin octapeptide, polymyxin heptapeptide or analogs and derivatives thereof. In preferred embodiments, the amount of polymyxin B is between about 800 U/g and about 80,000 U/g and the amount of magnesium sulfate is between 2 mg/g and 200 mg/g. In a more preferred embodiment, the amount of polymyxin B is between about 4,000 U/g and about 40,000 U/g and the amount of magnesium sulfate is about 100 mg/g. In another embodiment, the amount of polymyxin B nonapeptide can be between about 0.04 mg/ml and about 400 mg/ml and the amount of magnesium sulfate is between 2 mg/g and 200 mg/g. In a preferred embodiment, the amount of polymyxin B nonapeptide can be between about 0.4 mg/ml and about 40 mg/ml and the amount of magnesium sulfate is about 100 mg/g. The most preferred embodiments are Cream I which is a pharmaceutical composition comprising distilled water, polymyxin B, magnesium sulfate, glycerine, glyceryl dilaurate, glyceryl alcohol, ceteareth-20, isopropyl myristate, imidurea NF, guar gum, methyl pareben, and propyl paraben and Cream m which is pharmaceutical composition comprising distilled water, polymyxin B nonapeptide, magnesium sulfate, glycerine, glyceryl dilaurate, glyceryl alcohol, ceteareth-20, isopropyl myristate, imidurea NF, guar gum, methyl pareben, and propyl paraben. In a more preferred embodiment, Cream III comprises polymyxin B nonapeptide at a concentration of about 4 mg/ml.

Aminoglycoside Antibiotic Pharmaceutical Compositions:

The invention also includes a pharmaceutical composition for topical administration for localized chemodenervation comprising pharmaceutically effective amounts of an aminoglycoside antibiotic or analogs and derivatives thereof and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of aminoglycoside antibiotic or analogs and derivatives thereof and a magnesium salt or organic magnesium compound in a pharmaceutically acceptable medium compatible with human skin. In a further aspect, the invention includes a pharmaceutical composition for topical administration for reducing facial wrinkles comprising pharmaceutically effective amounts of aminoglycoside antibiotic or analogs and derivatives thereof and a magnesium salt or organic magnesium compound in a pharmaceutically acceptable medium compatible with human skin. In yet a further aspect, the invention includes a pharmaceutical composition for topical administration for treating or reducing neuropathic pain comprising pharmaceutically effective amounts of aminoglycoside antibiotic or analogs and derivatives thereof and a magnesium salt or organic magnesium compound in a pharmaceutically acceptable medium compatible with human skin.

The aminoglycoside antibiotic can be neomycin and the magnesium salt can be magnesium sulfate. In preferred embodiments, the amount of neomycin is between about 2.5 mg/g and 250 mg/g and the amount of magnesium sulfate is between 2 mg/g and 200 mg/g. In a more preferred embodiment, the amount of neomycin can be about 125 mg/g and the amount of magnesium sulfate can be about 100 mg/g. The most preferred embodiment is Cream II which is a pharmaceutical composition comprising distilled water, neomycin, magnesium sulfate, glycerine, glyceryl dilaurate, glyceryl alcohol, ceteareth-20, isopropyl myristate, imidurea NF, guar gum, methyl pareben, and propyl paraben.

Tetracycline Pharmaceutical Compositions:

The invention also includes a pharmaceutical composition for topical administration for localized chemodenervation comprising pharmaceutically effective amounts of an tetracycline or analogs and derivatives thereof and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of tetracycline or analogs and derivatives thereof and a magnesium salt or organic magnesium compound in a pharmaceutically acceptable medium compatible with human skin. In a further aspect, the invention includes a pharmaceutical composition for topical administration for reducing facial wrinkles comprising pharmaceutically effective amounts of tetracycline or analogs and derivatives thereof and a magnesium salt or organic magnesium compound in a pharmaceutically acceptable medium compatible with human skin. In yet a further aspect, the invention includes a pharmaceutical composition for topical administration for treating or reducing neuropathic pain comprising pharmaceutically effective amounts of tetracycline or analogs and derivatives thereof and a magnesium salt or organic magnesium compound in a pharmaceutically acceptable medium compatible with human skin. The magnesium salt can be magnesium sulfate.

Lincosamide Pharmaceutical Compositions:

The invention also includes a pharmaceutical composition for topical administration for localized chemodenervation comprising pharmaceutically effective amounts of an lincosamide or analogs and derivatives thereof and a magnesium salt or organic magnesium compound in a pharmaceutically acceptable medium compatible with human skin. In a another aspect, the invention includes a pharmaceutical composition for topical administration for treating involuntary facial muscle spasms comprising pharmaceutically effective amounts of lincosamide or analogs and derivatives thereof and a magnesium salt or organic magnesium compound in a pharmaceutically acceptable medium compatible with human skin. In a further aspect, the invention includes a pharmaceutical composition for topical administration for reducing facial wrinkles comprising pharmaceutically effective amounts of lincosamide or analogs and derivatives thereof and a magnesium salt or organic magnesium compound in a pharmaceutically acceptable medium compatible with human skin. In yet a further aspect, the invention includes a pharmaceutical composition for topical administration for treating or reducing neuropathic pain comprising pharmaceutically effective amounts of lincosamide or analogs and derivatives thereof and a magnesium salt or organic magnesium compound in a pharmaceutically acceptable medium compatible with human skin. The lincosamide can be clindamycin, lincomycin or analogs and derivatives thereof. The magnesium salt can be magnesium sulfate.

Muscle Relaxant Pharmaceutical Compositions:

The invention also includes a pharmaceutical composition for topical administration for localized chemodenervation comprising pharmaceutically effective amounts of an muscle relaxant or analogs and derivatives thereof and a magnesium salt or organic magnesium compound in a pharmaceutically acceptable medium compatible with human skin. In another aspect, the invention includes a pharmaceutical composition for topical administration for treating involuntary facial muscle spasms comprising pharmaceutically effective amounts of muscle relaxant or analogs and derivatives thereof and a magnesium salt or organic magnesium compound in a pharmaceutically acceptable medium compatible with human skin. In a further aspect, the invention includes a pharmaceutical composition for topical administration for reducing facial wrinkles comprising pharmaceutically effective amounts of muscle relaxant or analogs and derivatives thereof and a magnesium salt or organic magnesium compound in a pharmaceutically acceptable medium compatible with human skin. In yet a further aspect, the invention includes a pharmaceutical composition for topical administration for treating or reducing neuropathic pain comprising pharmaceutically effective amounts of muscle relaxant or analogs and derivatives thereof and a magnesium salt or organic magnesium compound in a pharmaceutically acceptable medium compatible with human skin. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. The magnesium salt can be magnesium sulfate.

Plant/Herb Extract Pharmaceutical Compositions:

The invention also includes a pharmaceutical composition for topical administration for localized chemodenervation comprising pharmaceutically effective amounts of an plant/herb extract or analogs and derivatives thereof and a magnesium salt or organic magnesium compound in a pharmaceutically acceptable medium compatible with human skin. In another aspect, the invention includes a pharmaceutical composition for topical administration for treating involuntary facial muscle spasms comprising pharmaceutically effective amounts of plant/herb extract or analogs and derivatives thereof and a magnesium salt or organic magnesium compound in a pharmaceutically acceptable medium compatible with human skin. In a further aspect, the invention includes a pharmaceutical composition for topical administration for reducing facial wrinkles comprising pharmaceutically effective amounts of plant/herb extract or analogs and derivatives thereof and a magnesium salt or organic magnesium compound in a pharmaceutically acceptable medium compatible with human skin. In yet a further aspect, the invention includes a pharmaceutical composition for topical administration for treating or reducing neuropathic pain comprising pharmaceutically effective amounts of plant/herb extract or analogs and derivatives thereof and a magnesium salt or organic magnesium compound in a pharmaceutically acceptable medium compatible with human skin.

The plant extract can be toosendanin, coryneine, banana truck extract, curare, black cohosh or analogs and derivatives thereof and the magnesium salt can be magnesium sulfate. In preferred embodiments, the amount of plant extract is between about 0.01 ml and about 1 ml of plant extract per gram of base cream and the amount of magnesium sulfate is between 2 mg/g and 200 mg/g (about 1 ml of plant extract (i.e. black cohosh) can be derived from about 2.3 g of dried herb). In a more preferred embodiment, the amount of plant extract is between about 0.01 ml and about 1 ml of plant extract per gram of base cream and the amount of magnesium sulfate can be about 100 mg/g (about 1 ml of plant extract (i.e. black cohosh) can be derived from about 2.3 g of dried herb). In a more preferred embodiment, the plant extract is black cohosh. The most preferred embodiment for Cream IV is a formulation comprising distilled water, black cohosh, magnesium sulfate, glycerine, glyceryl dilaurate, glyceryl alcohol, ceteareth-20, isopropyl myristate, imidurea NF, guar gum, methyl pareben, and propyl paraben. In a preferred embodiment, the concentration of black cohosh is 0.1 ml extract per gram of the base cream for the formulation of Cream IV. About 1 ml of black cohosh for use in the formulation of Cream IV can be derived from about 2.3 g of dried herb

Combinational Pharmaceutical Compositions:

The compounds of the present invention, e.g., antibiotics (including but not limited to: aminoglycoside antibiotics, tetracyclines, polymyxin (including but not limited to polymyxin B and polymyxin B nonapeptide), lincosamides), magnesium and muscle relaxants, plant extracts (including but not limited to: toosendanin, coryneine, banana truck extract, curare or black cohosh), (also referred to herein as “active compounds”) and derivatives, fragments, analogs and homologs thereof, can be incorporated into pharmaceutical compositions suitable for administration which can then be utilized in combination with other known compounds for localized chemodenervation, treating involuntary facial muscle spasms, reducing facial wrinkles or treating or reducing neuropathic pain. Theses compounds are used either to increase the penetration of active ingredients through the skin or to increase the release of active ingredients from the formulation to the skin. These compounds known in the field include, but are not limited to, azone, certain surfactants, DMSO, alcohol, acetone, propyleneglycol and polyethylene glycol. In addition, various drug delivery systems, including liposome and microscopic polymer-based microspares, may be applied to control the rate of drug delivery to the skin or to allow the skin to control the rate of drug absorption. In preferred embodiments, the compounds of the present invention, e.g., antibiotics (including but not limited to: aminoglycoside antibiotics, tetracyclines, polymyxin (including but not limited to polymyxin B and polymyxin B nonapeptide), and lincosamides), magnesium and muscle relaxants, plant or herb extracts and derivatives, fragments, analogs and homologs thereof are utilized in combination with other compounds of the present invention (e.g., aminoglycoside antibiotics in combination with magnesium, polymyxins in combination with muscle relaxants etc.) for localized chemodenervation, treating involuntary facial muscle spasms, reducing facial wrinkles or treating or reducing neuropathic pain. In preferred embodiments, two or more compounds are utilized in combination.

Kits:

The pharmaceutical compositions can be included in a kit, container, pack, or dispenser together with instructions for administration. In one aspect, the invention includes a kit comprising any of the pharmaceutical compositions for localized chemodenervation described herein and instructions for topical administration of the composition. The pharmaceutical composition can be antibiotics (including but not limited to: aminoglycoside antibiotics, tetracyclines, polymyxin (including but not limited to polymyxin B and polymyxin B nonapeptide), and lincosamides), magnesium, plant and herb extracts and muscle relaxants or analogs and derivatives thereof. The aminoglycoside antibiotic can be amikacin, gentamicin, kanamycin, neomycin, netilmicin, paromomycin, streptomycin, tobramycin or analogs and derivatives thereof. The lincosamide can be clindamycin, lincomycin or analogs and derivatives thereof. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt, an organic magnesium compound or analogs and derivatives thereof. The plant extract can be toosendanin, coryneine, banana truck extract, curare, black cohosh or analogs and derivatives thereof. In a preferred embodiment, the pharmaceutical composition can further comprise a magnesium salt or an organic magnesium compound.

In another aspect, the invention includes a kit comprising any of the pharmaceutical compositions for treating involuntary muscle spasms described herein and instructions for topical administration of the composition. The pharmaceutical composition can be antibiotics (including but not limited to: aminoglycoside antibiotics, tetracyclines, polymyxin (including but not limited to polymyxin B and polymyxin B nonapeptide), and lincosamides), magnesium, plant and herb extracts and muscle relaxants or analogs and derivatives thereof. The aminoglycoside antibiotic can be amikacin, gentamicin, kanamycin, neomycin, netilmicin, paromomycin, streptomycin, tobramycin or analogs and derivatives thereof. The lincosamide can be clindamycin, lincomycin or analogs and derivatives thereof. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt, an organic magnesium compound or analogs and derivatives thereof. The plant extract can be toosendanin, coryneine, banana truck extract, curare, black cohosh or analogs and derivatives thereof. In a preferred embodiment, the pharmaceutical composition can further comprise a magnesium salt or an organic magnesium compound.

In yet another aspect, the invention includes a kit comprising any of the cosmetic compositions for treating facial wrinkles described herein and instructions for topical administration of the composition. The pharmaceutical composition can be antibiotics (including but not limited to: aminoglycoside antibiotics, tetracyclines, polymyxin (including but not limited to polymyxin B and polymyxin B nonapeptide), and lincosamides), magnesium, plant and herb extracts and muscle relaxants or analogs and derivatives thereof. The aminoglycoside antibiotic can be amikacin, gentamicin, kanamycin, neomycin, netilmicin, paromomycin, streptomycin, tobramycin or analogs and derivatives thereof. The lincosamide can be clindamycin, lincomycin or analogs and derivatives thereof. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt, an organic magnesium compound or analogs and derivatives thereof. The plant extract can be toosendanin, coryneine, banana truck extract, curare, black cohosh or analogs and derivatives thereof. In a preferred embodiment, the pharmaceutical composition can further comprise a magnesium salt or an organic magnesium compound.

In a further aspect, the invention includes a kit comprising any of the pharmaceutical compositions for treating or reducing neuropathic pain described herein and instructions for topical administration of the composition. The pharmaceutical composition can be antibiotics (including but not limited to: aminoglycoside antibiotics, tetracyclines, polymyxin (including but not limited to polymyxin B and polymyxin B nonapeptide), and lincosamides), magnesium, plant and herb extracts and muscle relaxants or analogs and derivatives thereof. The aminoglycoside antibiotic can be amikacin, gentamicin, kanamycin, neomycin, netilmicin, paromomycin, streptomycin, tobramycin or analogs and derivatives thereof. The lincosamide can be clindamycin, lincomycin or analogs and derivatives thereof. The muscle relaxant can be a non-depolarizing or depolarizing agent. In preferred embodiments, the non-depolarizing agent can be pancuronium, vecuronium, mivacurium, rocuronium or analogs and derivatives thereof and the depolarizing agent can be succinylcholine, decamethonium or analogs and derivatives thereof. In another preferred embodiment, the muscle relaxant is a magnesium salt, an organic magnesium compound or analogs and derivatives thereof. The plant extract can be toosendanin, coryneine, banana truck extract, curare, black cohosh or analogs and derivatives thereof. In a preferred embodiment, the pharmaceutical composition can further comprise a magnesium salt or an organic magnesium compound.

The invention will be further described in the following examples, which do not limit the scope of the invention described in the claims.

### EXAMPLES

### Example 1

### Stability Test of Cream I

To test stability, cream I was subjected to a stability test according to industry standard protocols. The cream was stored at 45° C. for twelve (12) weeks and the result was found to be satisfactory. While there was no emulsion separation at most of the stability stations, slight yellowing and subtle separation occurred at 45° C. between the sixth and twelfth week. However, this is far superior to the commercial magnesium sulfate cream, which manifested emulsion separation at 45° C. after three days. In addition, cream I underwent through five freeze/thaw cycles with no emulsion separation.

### Example 2

### Safety Test of Cream I

Cream I was subjected to a safety test according to industry standard protocols. To determine the irritation and/or sensitization potential of the cream I, a repeated insult patch test (RIPT) was carried out as follows. In the induction phase, a total of 9 Parke-Davis Readi-Bandage occlusive patches containing the cream were applied to the back of each subject over a period of 3 weeks. After a rest period of 2 weeks, a challenge patch was applied to a previously unpatched (virgin) test site (challenge phase). The site was scored 24 and 72 hours after the challenge.

A total of 55 subjects (9 males and 46 females, 18-67 years old) participated in the study. 54 subjects (54/55) satisfactorily completed the test, whereas one subject (1/55) discontinued for personal reasons unrelated to the study. Based on the scores ranging from 0 to 4 (0 being no evidence of any effect and 4 the most severe skin reactivity), there was no skin reactivity (Score 0) on any subjects at any time during the study. Cream I was “Dermatologist-Tested” and did not induce skin irritation nor show any evidence of induced allergic contact dermatitis.

### Example 3

### Topical Use of Cream I for Anti-Wrinkle Effect

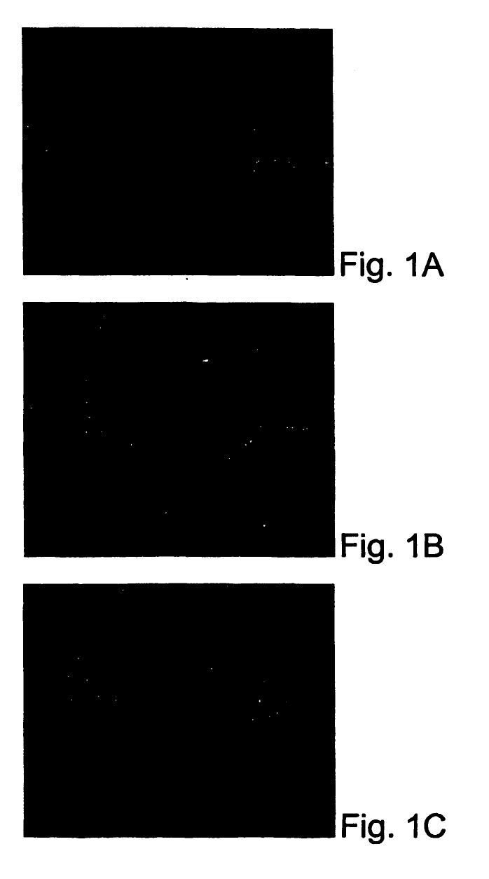

The efficacy of cream I was tested in clinical studies. The studies were approved by an independent institution review board (IRB). In a preliminary study, cream I was applied to the skin area around facial frowns. Pictures were taken pre-treatment and post-treatment on hourly basis. As shown in FIG. 1, facial frown lines were significantly reduced just two hours after applying cream I (Panel B) and kept improving by three hours (Panel C) as compared to untreated frown lines (Panel A). In addition, more smooth skin and relaxed muscle were observed after application (FIG. 1). Compared to Botox injection, topical use of cream I delivered its effect much faster and did not cause any numbness or stiffness.

Following the preliminary study, a phase I study was implemented to determine if the use of cream I decreased, within hours, the appearance of wrinkles at the forward/frown line area in a panel of 30 women (ages from 30-60 years). The cream was topically applied to the frown line area and the procedure repeated once an hour later. The evaluations, including clinician evaluation, photoimage and skin replica, took place between baseline (pre-application) up to 6 hours after initial cream application.

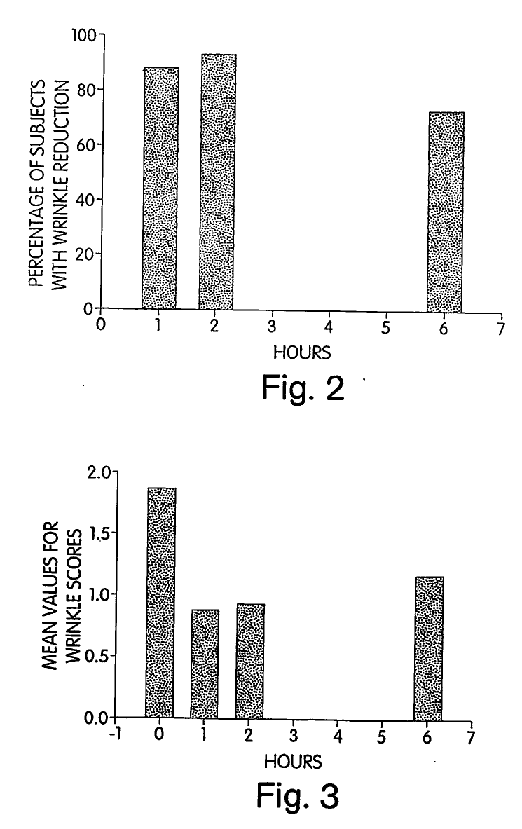

Clinician evaluation was based on a score system ranging from 0 to 3, 0 being smooth skin with no sign of wrinkles to 3 being visible and deep wrinkles. The data from the phase I study indicated that 88.5% of subjects were observed to have had visible reduction in frown lines one hour after cream application (FIG. 2). At the 6 hour time point, the percentage of subjects with visible wrinkle reduction remained approximately 70% (FIG. 2). The mean value for the wrinkle score was decreased from 1.87 to 0.88 (52.9% reduction) within one hour after the application of the cream. At the 6 hour time point, the mean value was 1.17 (37.4% reduction from the baseline) (FIG. 3). The mean values for wrinkle scores were calculated based on 30 subjects at each time point except at 1 hour (26 subjects). These changes were statistically significant at every time points tested (1, 2 and 6 hours). Skin replica was analyzed for the skin texture, measured by eight parameters. Rz and Ra are optical counterparts of classic “stylus” roughness texture parameters. Fspace is the distance between markers in mm indicative of fine and coarse lines. FNum is number markers per mm. Spacing is the mean distance in millimeters between adjacent strong shadow features. Breadth is proportional to the depth of the wrinkle producing the shadow. Shadows parameter is the relative area of shadows cast by all the wrinkles and fine lines in the replica. NumWr is the total number of shadowy features available to calculate spacing and breadth. Depending on the direction of the lighting source, the skin replica can be analyzed in both normal orientation (sensitive to the longitudinal lines) and parallel orientation (sensitive to the lateral lines running “ear to ear”). Analysis was based on the data derived from the normal orientation as the frown lines were the focus of the study.

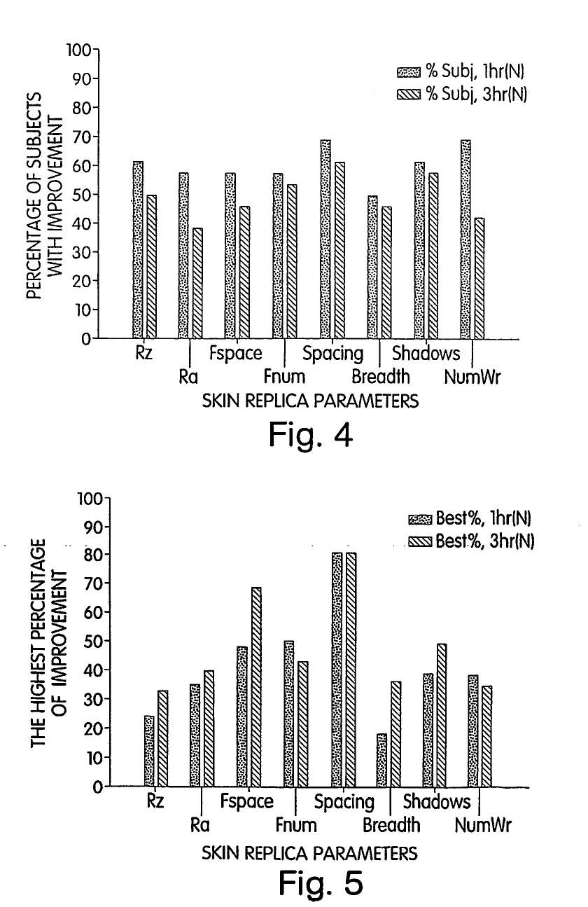

Based on eight different parameters measuring the skin texture, a quantitative improvement was observed in 50.0%-69.2% of subjects after 1 hour and 38.5%-61.5% of subjects after 3 hours (FIG. 4). The best improvement (%) in each parameter ranged from 18.9%-81.5% after 1 hour and 33.3%-81.2% after 3 hours (FIG. 5). The majority of subjects (57.7%) demonstrated improvement in at least 6 of 8 parameters, although the mean changes were not statistically significant. Visual improvement was observed in most subjects in the study.

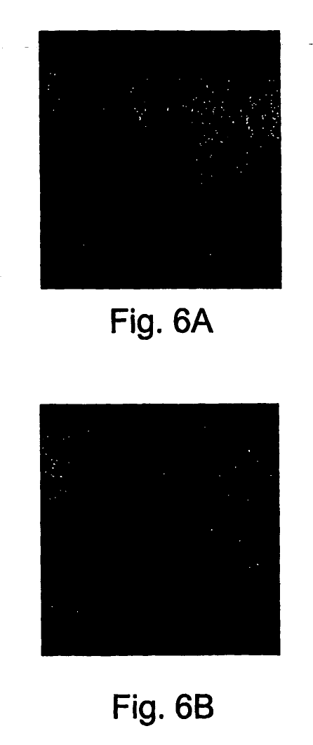

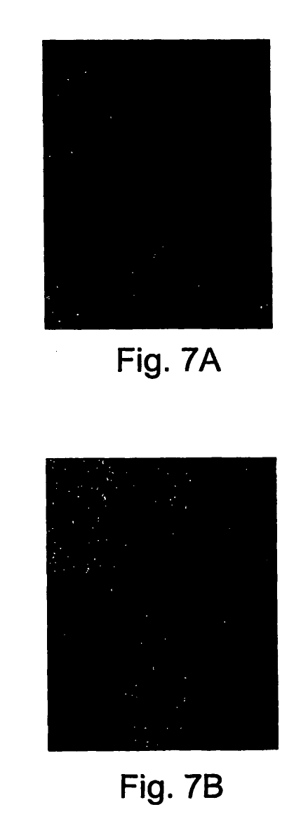

As shown in FIG. 6, facial frown lines of a 55 year old female patient (Panel A) were reduced just one hour after the treatment with cream I (Panel B). Similar results are shown in FIG. 7, the facial frown lines of a 57 year old female patient (Panel A) were reduced one hour after treatment with cream I (Panel B).

### Example 4

### Topical Use of Cream I for Treating Synkinesis

Cream I was tested in a patient with synkinesis. The patient, a 44 years old male, had surgery for acoustic neuroma two years ago. During the recovery from facial paralysis, he developed minor synkinesis. When he tried to pucker and stretch his mouth, involuntary muscle contraction under his right eye was observed (left eye is perfectly normal).

Cream I was applied to the area under the patient's right eye. In 10-15 minutes, the involuntary muscle contraction was significantly reduced. By 30 minutes, the synkinesis was almost completely gone. The effect lasted 5-6 hours. After that, the synkinesis slowly returned.

The therapeutic effect of cream I results from the presence of polymyxin B or polymyxin derivative, which demonstrates a dose-response property: while a control cream, containing no polymyxin B or polymyxin derivative, did not show any activity, the inhibition of synkinesis became more obvious as the concentration of polymyxin B or polymyxin derivative increased (4,000, 10,000, 20,000 and 40,000 U/g were tested).

On the other hand, polymyxin B alone (an over-the-counter drug containing 10,000 U/g polymyxin B was tested) did not show any inhibition of synkinesis, thereby suggesting the importance of magnesium, another neuromuscular blocking agent in the formulation.

### Example 5

### Topical Use of Cream II for Anti-Wrinkle Effect

In order to demonstrate that other compounds of the instant invention may work in a similar fashion as Cream I, Cream II was formulated using the aminoglycoside, neomycin, and was applied to frown lines. After one hour, frown lines were visibly reduced. Cream II can be applied again after 0.5-1 hour to achieve a better result.

### Example 6

### Animal Model

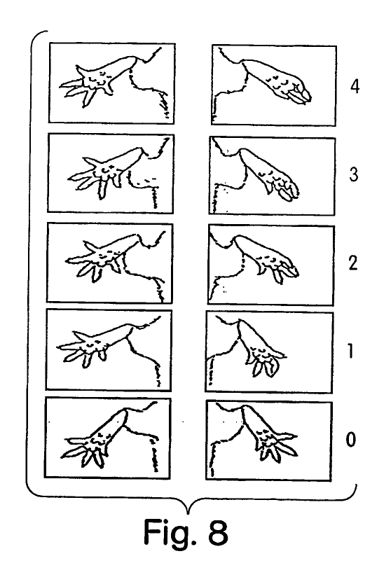

Based on a procedure used by Allergan for analysis of Botox product (Aoki, Toxicon 39: 1815-1820, 2001), a mouse model was established to examine the neuromuscular blockade potential of various compounds (antibiotics (including but not limited to: aminoglycoside antibiotics, tetracyclines, polymyxin (including but not limited to polymyxin B and polymyxin B nonapeptide), and lincosamides), magnesium, plant and herb extracts and muscle relaxants or analogs and derivatives thereof) described herein. After a compound was topically applied to the gastrocnemius muscle of mouse legs, its effect could be semi-quantitatively measured by briefly suspending the tail to elicit a characteristic startle response in which the mouse extends its hind limbs and abducts its hind digits (FIG. 8). The left half panels of FIG. 8 show the non-injected side and the right half panels the injected side of the mouse leg. A scale from 0 to 4 is used to measure the muscle weakness: a reading 0 indicates normal while a reading 4 represents maximal muscle weakening (Aoki, K. R. Toxicon, 39 (Aoki et al. Toxicon 39: 1815-1820, 2001).

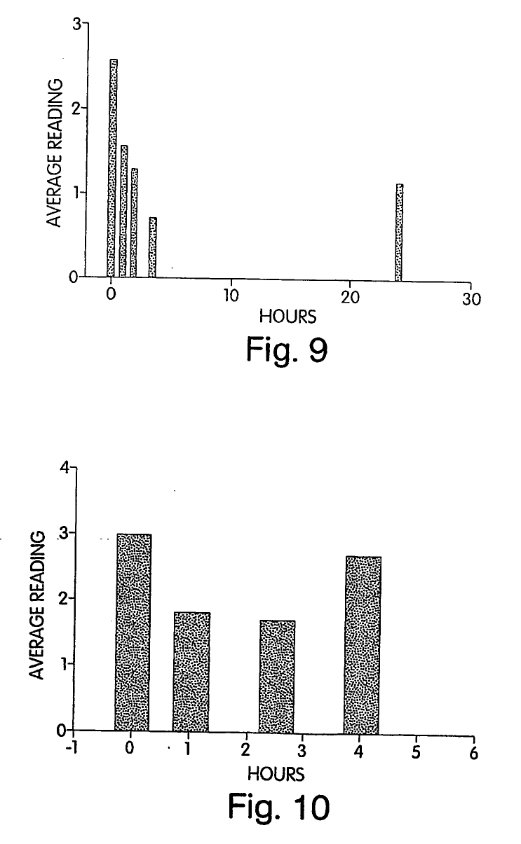

For example, the antagonistic effect of 3,4-diaminopyridine (DAP) was tested on Botox by topical administration. DAP belongs to a group of compounds that act on nerve membranes to promote influx of calcium, which in turn greatly promotes efflux of acetylcholine. In a group of mice (n=7), average reading after Botox treatment is 2.5 (in a scale from 0 to 4, 0 being normal and 4 being most severe). After topically applying DAP on the legs, the reading was recorded as 1.5 at 1 hour and continued to drop to 0.75 at 4 hour (FIG. 9).

In a separate experiment (mouse number=6), rapid response to topical administration of DAP was observed, although the reading went up after 4 hours of the treatment (FIG. 10). This study indicates that this mouse model can be effectively used to test and screen compounds, by topical administration, for activities of modulating neuromuscular transmission.

### Example 7

### Preparation of Polymyxin B Nonapeptide (PMBN)

Polymyxin B nonapeptide (PMBN) was prepared by modification of published procedures known in the art. Polymyxin B (1 g, purchased from Voigt Global Distribution LLC) was dissolved in 20 ml of deionized water in a 50-ml tube. A papain solution was prepared by adding 125 mg of papain (from papaya latex, distributed by Sigma-aldrich) in 4 ml of deionized water. To the 50-ml tube, 2 ml of the papain solution was added. After incubation at 37° C. with a constant rotation (50 rpm) for 5 hrs, another 1 ml of the papain solution was added to the tube. After overnight digestion, 1 ml of the papain solution was added and incubated at 25° C. with constant rotation for 24 hrs. The total incubation time is 48 hrs.

To remove papain from the digested Polymyxin B solution, the 50-ml tube was placed in a 95° C. water bath for 5 min, followed by sitting on ice for 10 min. The heat-denatured papain was then removed by centrifugation at 10,000 rpm (15,000 g, SLA-1500 rotor) for 20 min. The supernatant containing PMBN (25 ml) was stored at −20° C. for further analysis.

### Example 8

### Characterization of Polymyxin B Nonapeptide (PMBN)

Chemical Characterization of Polymyxin B nonapeptide (PMBN)

The prepared PMBN can be analyzed by HPLC, thin-layer chromatography, UV spectrum, or amino acid analysis. The controls include the starting material, the PMB solution, and commercially obtained PMBN.

Analytical reversed-phase HPLC is performed using a column (150×6 mm, i.d.), isocratic elution with 0.01N H2SO4 in MeOH—H2O (35:65), the flow rate of 0.9 ml/min and the detection and 210 nm. The thin-layer-chromatography is performed on cellulose plate with butanol-pyridine-acetic acid-water (30:20:6:24, in volume).

Characterization of the Loss of Antibacterial Activity of Polymyxin B nonapeptide (PMBN)

The loss of antibacterial activity of PMBN can be verified by a standard bacteria inhibition assay. Briefly, an E. coli overnight culture is placed on the top of an agar petri-dish. Small holes are punched on the agar. After adding the PMBN solution (20 ul) and other controls to the holes, the plate is placed in a 37° C. incubator overnight. The controls include the PMB solution (starting material) and commercially obtained PMBN. Peptides with antibacterial activity (if any) can diffuse into the agar and inhibit the growth of E. coli on the surface around the holes. The antibacterial potency correlates with the diameter of the ring where bacterial growth is inhibited.

### Example 9

### Topical Use of Cream m for Treating Synkinesis

The patient, a 44 years old male, had surgery for acoustic neuroma two years ago. During the recovery from facial paralysis, he developed minor synkinesis. When he tried to pucker and stretch his mouth, involuntary muscle contraction under his right eye was observed (left eye is perfectly normal).

Cream III was applied to the area under the patient's right eye and the patient was examed every hour. At 1 hour, synkinesis was significantly suppressed. The suppression was continually observed up to the 6-hour time point. At 7 hour, some synkinesis was observed. After that, the synkinesis slowly returned.

As a dose-response study, the cream containing 2 mg/ml PMBN was applied in the same manner as described above. Synkinesis was largely reduced in an hour. However, at 3 hour, the suppression started to fade. The cream containing 1 mg/ml PMBN had almost no effect on synkinesis as examined at 1 and 2 hours after the cream application. As a negative control, the cream without PMBN did not result in any visible changes on the patient's face.

### Example 10

### Topical Use of Cream III for Anti-Wrinkle Effect

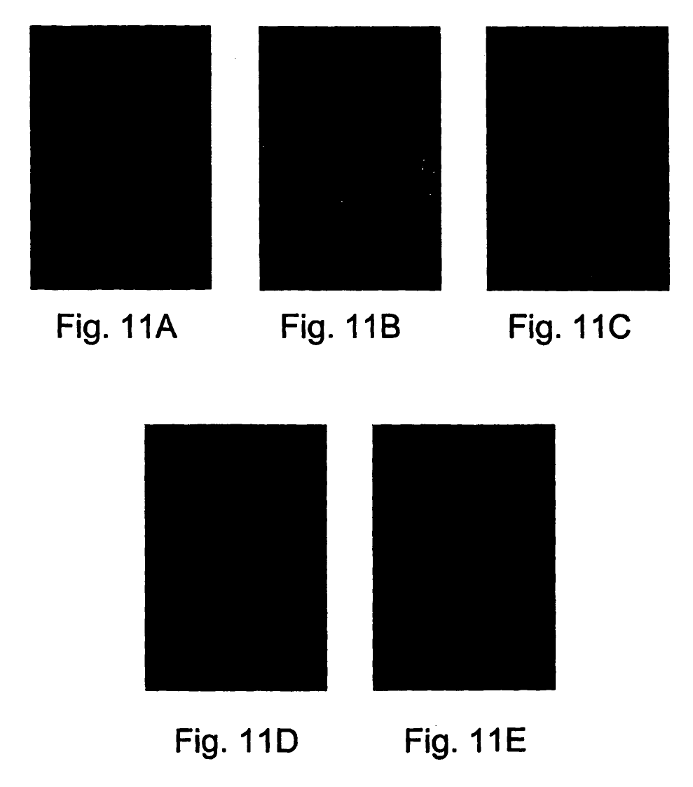

Cream III was applied to the skin area around facial frowns. Pictures were taken pre-treatment and post-treatment. As shown in the FIG. 11, cream III was applied to untreated frown lines (Panel A). After applying cream III, facial frown lines were significantly reduced just 1 hour after the treatment (Panel B). After 1 hour of treatment, cream III was applied again and the results show greater reduction in frown lines after two hours (Panel C). However, when untreated frown lines (Panel D) were treated a cream which did not contain PMBN no visible effect on the frown lines were observed (Panel E).

### Example 11

### Preparation Of Plant Extracts with Neuromuscular Blocking Activity

Plants and/or herbs were ground and extracted with distilled water. After boiling for 10 minutes and cooling down to room temperature overnight, the extracts were separated from any insoluble fraction by centrifugation at 5,000 rpm for five minutes. The extracts were then concentrated on a rotary evaporator and stored at −20° C. for testing in an animal model or human volunteers.

| | | Dry | Extraction | Final |
|---|---|---|---|---|
| | Active | weight | volume | volume |
| Plant/herb | ingredient | (g) | (ml) | (ml) |
| Fructus Meliae | Toosendanin | 33.8 | 250 | 18 |
| Aconite Root | coryneine | 22 | 150 | 16 |
| Cortex Meliae | Toosendanin | 19 | 150 | 12 |
| Black Cohosh | ciminifuga | 30 | 440 | 13 |
| | racemose | | | |
| Passion Flower | passiflora | 36 | 570 | 25 |
| | incarnata | | | |
| Kava | piper | 47 | 650 | 26 |
| | methysticum | | | |

### Example 12

### Preparation of Black Cohosh Extract

Black cohosh extract was prepared from the black cohosh root tea bags (purchased from Alvita Herbal Teas, American Fork, Utah 84003). Black cohosh root (30 g) was mixed with 200 ml of water. After boiling for 10 min, the mixture was kept at 70° C. for 2 hours. After overnight at 4° C., the mixture was then spun at 5,000 rpm (SLA-1500 rotor) for 5 min. The insoluble herb was extracted again with 200 ml of water according to the above procedure. The supernatants from two centrifugations were combined and concentrated to the final volume of 13 ml by using a rotary evaperator at 48° C. The extract was stored at −20° C.

### Example 13

### Topical Use of Cream IV for Treating Synkinesis

The patient, a 44 years old male, had surgery for acoustic neuroma two years ago. During the recovery from facial paralysis, he developed minor synkinesis. When he tried to pucker and stretch his mouth, involuntary muscle contraction under his right eye was observed (left eye is perfectly normal).

Cream IV was applied to the area under the patient's right eye and the patient was examined every hour. At 1 hour, synkinesis was significantly suppressed. The suppression was continually observed up to the 8-hour time point. After 9 hours, synkinesis slowly returned.

As a control, a cream without black cohosh did not result in any visible changes on the patient face.

Phytoestrogen Body Cream (Transitions for Health, Inc. Portland, Oreg. 97205) was also tested, following the same procedure described above. The Phytoestrogen Body Cream failed to show any activity in suppressing the symptoms of synkinesis.

### Example 14

### Topical Use of Cream IV for Anti-Wrinkle Effect

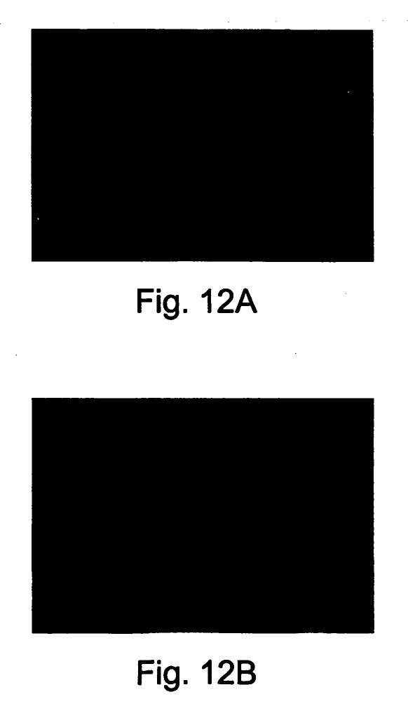

Cream IV was applied to the skin area around facial frowns. Pictures were taken pre-treatment and post-treatment. As shown in FIG. 12, facial frown lines (Panel A) were significantly reduced two hours after the treatment with Cream IV (Panel B). However, a cream which did not contain black cohosh did not result in any visible effect on the frown lines.

### OTHER EMBODIMENT

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for localized chemodenervation comprising topically administering a pharmaceutical composition comprising a pharmaceutically effective amount of an antibiotic and a pharmaceutically acceptable carrier.

2. A method for localized chemodenervation comprising topically administering a pharmaceutical composition comprising a pharmaceutically effective amount of an aminoglycoside antibiotic or an analog or derivative thereof and a pharmaceutically acceptable carrier.

3. The method of claim 2, wherein the aminoglycoside antibiotic is selected from the group consisting of amikacin, gentamicin, kanamycin, neomycin, netilmicin, paromomycin, streptomycin and tobramycin.

4. The method of claim 2, wherein the pharmaceutical composition further comprises a magnesium salt or an organic magnesium compound.

5. A method for localized chemodenervation comprising topically administering a pharmaceutical composition comprising a pharmaceutically effective amount of a polymyxin or an analog or derivative thereof and a pharmaceutically acceptable carrier.

6. The method of claim 5, wherein the polymyxin is polymyxin B or polymyxin B nonapeptide.

7. The method of claim 5, wherein the pharmaceutical composition further comprises a magnesium salt or an organic magnesium compound.

8. A method for localized chemodenervation comprising topically administering a pharmaceutical composition for comprising a pharmaceutically effective amount of tetracycline or an analog or derivative thereof and a pharmaceutically acceptable carrier.

9. A method for localized chemodenervation comprising topically administering a pharmaceutical composition comprising a pharmaceutically effective amount of lincosamide or an analog or derivative thereof and a pharmaceutically acceptable carrier.

10. The method of claim 9 wherein the lincosamide is clindamycin or lincomycin.

11. A method for localized chemodenervation comprising topically administering a pharmaceutical composition comprising a pharmaceutically effective amount of a muscle relaxant or an analog or derivative thereof and pharmaceutically acceptable carrier.

12. The method of claim 11 wherein the muscle relaxant is a non-depolarizing agent.

13. The method of claim 12 wherein the non-depolarizing agent is pancuronium, vecuronium, mivacurium or rocuronium.

14. The method of claim 11 wherein the muscle relaxant is a depolarizing agent.

15. The method of claim 14 wherein the depolarizing agent is succinylcholine or decamethonium.

16. The method of claim 11 wherein the muscle relaxant is a magnesium salt or an organic magnesium compound.

17. A method for localized chemodenervation comprising topically administering a pharmaceutical composition comprising a pharmaceutically effective amount of a plant extract or analog or derivative thereof, and a pharmaceutically acceptable carrier.

18. The method of claim 17, wherein the plant extract is toosendanin, coryneine, banana truck extract, or curare or black cohosh.

19. The method of claim 17, wherein the pharmaceutical composition further comprises a magnesium salt or an organic magnesium compound.

20. A method for treating involuntary facial muscle spasms comprising topically administering a pharmaceutical composition comprising a pharmaceutically effective amount of composition selected from the group consisting of aminoglycoside antibiotic, polymyxin, tetracycline, lincosamide, muscle relaxant and plant extract, or an analog or derivative thereof and a pharmaceutically acceptable carrier.

21. The method of claim 20, wherein the aminoglycoside antibiotic is selected from the group consisting of amikacin, gentamicin, kanamycin, neomycin, netilmicin, paromomycin, streptomycin and tobramycin.

22. The method of claim 20, wherein the polymyxin is polymyxin B or polymyxin B nonapeptide.

23. The method of claim 20, wherein the plant extract is toosendanin, coryneine, banana truck extract, curare or black cohosh.

24. The method of claim 20, wherein the pharmaceutical composition further comprises a magnesium salt or an organic magnesium compound.

25. The method of claim 20 wherein the involuntary facial muscle spasms are caused by synkinesis, ocular disorders, dystonia, hemifacial spasm, or blepharospasm.

26. A method for reducing facial wrinkles comprising topically administering a pharmaceutical composition comprising a pharmaceutically effective amount of composition selected from the group consisting of aminoglycoside antibiotic, polymyxin, tetracycline, lincosamide, muscle relaxant and plant extract, or an analog or derivative thereof and a pharmaceutically acceptable carrier.

27. The method of claim 26, wherein the aminoglycoside antibiotic is selected from the group consisting of amikacin, gentamicin, kanamycin, neomycin, netilmicin, paromomycin, streptomycin and tobramycin.

28. The method of claim 26, wherein the polymyxin is polymyxin B or polymyxin B nonapeptide.

29. The method of claim 26, wherein the plant extract is toosendanin, coryneine, banana truck extract, curare or black cohosh.

30. The method of claim 26, wherein the pharmaceutical composition further comprises a magnesium salt or an organic magnesium compound.

31. A method for treating or reducing neuropathic pain comprising topically administering a pharmaceutical composition comprising a pharmaceutically effective amount of composition selected from the group consisting of aminoglycoside antibiotic, polymyxin, tetracycline, lincosamide, muscle relaxant and plant extract, or an analog or derivative thereof and a pharmaceutically acceptable carrier.

32. The method of claim 31, wherein the aminoglycoside antibiotic is selected from the group consisting of amikacin, gentamicin, kanamycin, neomycin, netilmicin, paromomycin, streptomycin and tobramycin.

33. The method of claim 31, wherein the polymyxin is polymyxin B or polymyxin B nonapeptide.

34. The method of claim 31, wherein the plant extract is toosendanin, coryneine, banana truck extract, curare or black cohosh.

35. The method of claim 31, wherein the pharmaceutical composition further comprises a magnesium salt or an organic magnesium compound.

36. The method of claim 31 wherein the neuropathic pain is caused by postherpetic neuralgia, diabetic neuropathy, complex regional pain syndrome, spinal cord injury, radiculopathy, migraine headache, myofascial pain, or carpal tunnel syndrome.

37. A pharmaceutical composition for topical administration for localized chemodenervation comprising pharmaceutically effective amounts of
a polymyxin or an analog or derivative thereof and
a magnesium salt or organic magnesium compound
in a pharmaceutically acceptable medium compatible with human skin.

38. The method of claim 37 wherein the polymyxin is polymyxin B or polymyxin B nonapeptide.

39. The pharmaceutical composition of claim 38 wherein the amount of polymyxin B is between about 800 U/g and about 80,000 U/g.

40. The pharmaceutical composition of claim 38 wherein the amount of polymyxin B nonapeptide is between about 0.04 mg/ml and about 400 mg/ml.

41. The pharmaceutical composition of claim 37 wherein the magnesium salt is magnesium sulfate.

42. The pharmaceutical composition of claim 41 wherein the amount of magnesium sulfate is between about 2 mg/g and about 200 mg/g.

43. A pharmaceutical composition for topical administration for localized chemodenervation comprising pharmaceutically effective amounts of
an aminoglycoside antibiotic or an analog or derivative thereof and
a magnesium salt or organic magnesium compound
in a pharmaceutically acceptable medium compatible with human skin.

44. The pharmaceutical composition of claim 43 wherein the aminoglycoside antibiotic is selected from the group consisting of amikacin, gentamicin, kanamycin, neomycin, netilmicin, paromomycin, streptomycin and tobramycin.

45. The pharmaceutical composition of claim 44 wherein the amount of neomycin is between about 2.5 mg/g and about 200 mg/g.

46. The pharmaceutical composition of claim 43 wherein the magnesium salt is magnesium sulfate.

47. The pharmaceutical composition of claim 46 wherein the amount of magnesium sulfate is between about 2 mg/g and about 200 mg/g.

48. A pharmaceutical composition for topical administration for localized chemodenervation comprising pharmaceutically effective amounts of
plant extract or an analog or derivative thereof and
a magnesium salt or organic magnesium compound
in a pharmaceutically acceptable medium compatible with human skin.

49. The pharmaceutical composition of claim 48 wherein the plant extract is toosendanin, coryneine, banana truck extract, curare or black cohosh.

50. The pharmaceutical composition of claim 49 wherein the amount of black cohosh is between about 0.01 ml/g and about 1 ml/g.

51. The pharmaceutical composition of claim 48 wherein the magnesium salt is magnesium sulfate.

52. The pharmaceutical composition of claim 51 wherein the amount of magnesium sulfate is between about 2 mg/g and about 200 mg/g.

53. A pharmaceutical composition for topical administration for localized chemodenervation comprising a pharmaceutically effective amount of tetracycline or an analog or derivative thereof and in a pharmaceutically acceptable medium compatible with human skin.

54. The pharmaceutical composition of claim 53 further comprising a magnesium salt or organic magnesium compound.

55. The pharmaceutical composition of claim 54 wherein the magnesium salt is magnesium sulfate.

56. A pharmaceutical composition for topical administration for localized chemodenervation comprising a pharmaceutically effective amount of lincosamide or an analog or derivative thereof in a pharmaceutically acceptable medium compatible with human skin.

57. The pharmaceutical composition of claim 56 wherein the lincosamide is clindamycin or lincomycin.

58. The pharmaceutical composition of claim 56 further comprising a magnesium salt or organic magnesium compound.

59. The pharmaceutical composition of claim 58 wherein the magnesium salt is magnesium sulfate.

60. A pharmaceutical composition for topical administration for localized chemodenervation comprising pharmaceutically effective amount of a muscle relaxant or an analog or derivative thereof in a pharmaceutically acceptable medium compatible with human skin.

61. The pharmaceutical composition of claim 60 wherein the muscle relaxant is a non-depolarizing agent.

62. The pharmaceutical composition of claim 61 wherein the non-depolarizing agent is pancuronium, vecuronium, mivacurium or rocuronium.

63. The pharmaceutical composition of claim 60 wherein the muscle relaxant is a depolarizing agent.

64. The pharmaceutical composition of claim 63 wherein the depolarizing agent is succinylcholine or decamethonium.

65. The pharmaceutical composition of claim 60 wherein the muscle relaxant is a magnesium salt or an organic magnesium compound.

66. The pharmaceutical composition of claim 60 further comprising a magnesium salt or organic magnesium compound.

67. The pharmaceutical composition of claim 66 wherein the magnesium salt is magnesium sulfate.

68. A kit comprising the pharmaceutical composition of claim 37, 43, 48, 53, 56, or 60 and instructions on how to administer said pharmaceutical composition for localized chemodenervation.

69. A pharmaceutical composition for topical administration for treating involuntary facial muscle spasms comprising pharmaceutically effective amounts of a composition selected from the group consisting of aminoglycoside antibiotic, polymyxin, tetracycline, lincosamide, muscle relaxant and plant extract, or an analog or derivative thereof and a magnesium salt or organic magnesium compound in a pharmaceutically acceptable medium compatible with human skin.

70. The method of claim 69, wherein the polymyxin is polymyxin B or polymyxin B nonapeptide.

71. The pharmaceutical composition of claim 69 wherein the aminoglycoside antibiotic is selected from the group consisting of amikacin, gentamicin, kanamycin, neomycin, netilmicin, paromomycin, streptomycin and tobramycin.

72. The pharmaceutical composition of claim 69 wherein the plant extract is toosendanin, coryneine, banana truck extract, curare or black cohosh

73. The pharmaceutical composition of claim 69 wherein the magnesium salt is magnesium sulfate.

74. A kit comprising the composition of claim 69 and instructions on how to administer said composition to treat involuntary facial muscle spasms.

75. A pharmaceutical composition for topical administration for reducing facial wrinkles comprising pharmaceutically effective amounts of a composition selected from the group consisting of aminoglycoside antibiotic, polymyxin, tetracycline, lincosamide, muscle relaxant and plant extract, or an analog or derivative thereof and a magnesium salt or organic magnesium compound in a pharmaceutically acceptable medium compatible with human skin.

76. The method of claim 75, wherein the polymyxin is polymyxin B or polymyxin B nonapeptide.

77. The pharmaceutical composition of claim 75 wherein the aminoglycoside antibiotic is selected from the group consisting of amikacin, gentamicin, kanamycin, neomycin, netilmicin, paromomycin, streptomycin and tobramycin.

78. The pharmaceutical composition of claim 75 wherein the plant extract is toosendanin, coryneine, banana truck extract, curare or black cohosh

79. The pharmaceutical composition of claim 75 wherein the magnesium salt is magnesium sulfate.

80. A kit comprising the composition of claim 75 and instructions on how to administer said composition to reduce facial wrinkles.

81. A pharmaceutical composition for topical administration for treating or reducing neuropathic pain comprising pharmaceutically effective amounts of a composition selected from the group consisting of aminoglycoside antibiotic, polymyxin, tetracycline, lincosamide, muscle relaxant and plant extract, or an analog or derivative thereof and a magnesium salt or organic magnesium compound in a pharmaceutically acceptable medium compatible with human skin.

82. The method of claim 81, wherein the polymyxin is polymyxin B or polymyxin B nonapeptide.

83. The pharmaceutical composition of claim 81 wherein the aminoglycoside antibiotic is selected from the group consisting of amikacin, gentamicin, kanamycin, neomycin, netilmicin, paromomycin, streptomycin and tobramycin.

84. The pharmaceutical composition of claim 81 wherein the plant extract is toosendanin, coryneine, banana truck extract, curare or black cohosh

85. The pharmaceutical composition of claim 81 wherein the magnesium salt is magnesium sulfate.

86. A kit comprising the composition of claim 81 and instructions on how to administer said composition to treat or reduce neuropathic pain.
